SEQUENCE LISTING (38 sequences)

SEQ ID NO: 1 - DNA, 1830 bp, Listeria monocytogenes
  CDS (1)..(1830)
gcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagaccaacatctgaagaatttagttcgctgaatagtggtgattttacagatgacgaaaacagcgagacaacagaagaagaaattgatcgcctagctgatttaagagatagaggaacaggaaaacactcaagaaatgcgggttttttaccattaaatccgtttgctagcagcccggttccttcgttaagtccaaaggtatcgaaaataagcgcaccggctctgataagtgacataactaaaaaaacgccatttaagaatccatcacagccattaaatgtgtttaataaaaaaactacaacgaaaacagtgactaaaaaaccaacccctgtaaagaccgcaccaaagctagcagaacttcctgccacaaaaccacaagaaaccgtacttagggaaaataaaacaccctttatagaaaaacaagcagaaacaaacaagcagtcaattaatatgccgagcctaccagtaatccaaaaagaagctacagagagcgataaagaggaaatgaaaccacaaaccgaggaaaaaatggtagaggaaagcgaatcagctaataacgcaaacggaaaaaatcgttctgctggcattgaagaaggaaaactaattgctaaaagtgcagaagacgaaaaagcgaaggaagaaccagggaaccatacgacgttaattcttgcaatgttagctattggcgtgttctctttaggggcgtttatcaaaattattcaattaagaaaaaataat

SEQ ID NO: 2 - protein, 610 aa, Listeria monocytogenes
ATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISAPALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 3 - DNA, 1128 bp, Listeria monocytogenes
  CDS (1)..(1128)
gacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagaccaacatctgaagaatttagttcgctgaatagtggtgattttacagatgacgaaaacagcgagacaacagaagaagaaattgatcgcctagctgatttaagagatagaggaacaggaaaacactcaagaaatgcgggttttttaccattaaatccgtttgctagcagcccggttccttcgttaagtccaaaggtatcgaaaataagcgcaccggctctgataagtgacataactaaaaaaacgccatttaagaatccatcacagccattaaatgtgtttaataaaaaaactacaacgaaaacagtgactaaaaaaccaacccctgtaaagaccgcaccaaagctagcagaacttcctgccacaaaaccacaagaaaccgtacttagggaaaataaaacaccctttatagaaaaacaagcagaaacaaacaagcagtcaattaatatgccgagcctaccagtaatccaaaaagaagctacagagagcgataaagaggaaatgaaaccacaaaccgaggaaaaaatggtagaggaaagcgaatcagctaataacgcaaacggaaaaaatcgttctgctggcattgaagaaggaaaactaattgctaaaagtgcagaagacgaaaaagcgaaggaagaaccagggaaccatacgacgttaattcttgcaatgttagctattggcgtgttctctttaggggcgtttatcaaaattattcaattaagaaaaaataat

SEQ ID NO: 4 - protein, 376 aa, Listeria monocytogenes
DFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISAPALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 5 - DNA, 1050 bp, Listeria monocytogenes
  CDS (1)..(1050)
gacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagaccaacatctgaagaatttagttcgctgaatagtggtgattttacagatgacgaaaacagcgagacaacagaagaagaaattgatcgcctagctgatttaagagatagaggaacaggaaaacactcaagaaatgcgggttttttaccattaaatccgtttgctagcagcccggttccttcgttaagtccaaaggtatcgaaaataagcgcaccggctctgataagtgacataactaaaaaaacgccatttaagaatccatcacagccattaaatgtgtttaataaaaaaactacaacgaaaacagtgactaaaaaaccaacccctgtaaagaccgcaccaaagctagcagaacttcctgccacaaaaccacaagaaaccgtacttagggaaaataaaacaccctttatagaaaaacaagcagaaacaaacaagcagtcaattaatatgccgagcctaccagtaatccaaaaagaagctacagagagcgataaagaggaaatgaaaccacaaaccgaggaaaaaatggtagaggaaagcgaatcagctaataacgcaaacggaaaaaatcgttctgctggcattgaagaaggaaaactaattgctaaaagtgcagaagacgaaaaagcgaaggaagaaccagggaaccatacgacg

SEQ ID NO: 6 - protein, 350 aa, Listeria monocytogenes
DFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISAPALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTT

SEQ ID NO: 7 - DNA, 1695 bp, Murine sp.
  CDS (1)..(1692)
atggcggccccccgcccgcctcccgcgatctccgtctccgtctcggcccccgcgttttacgccccgcagaagaagttcgccccggttgtggccccaaagcccaaagtgaatcctttccggcctggggacagcgagcctcctgtagcagccggggcccaaagagcgcagatgggtcgggtgggcgagatcccaccaccacccccggaagactttcctttgccccctcctccccttattggggagggcgacgactcagagggtgccctgggaggtgccttcccacctccacctcccccgatgatcgaggaaccattcccccctgctcctctggaggaggacatcttcccctcccctccacctccactggaggaggagggagggcctgaggcccctacccagctcccaccgcagcccagggagaaagtgtgcagtattgacctggagattgactctctgtcctcactgctggacgacatgaccaagaacgatcccttcaaagcccgggtatcatccggatatgtacccccaccagttgccactccatttgttcccaagcctagtaccaaacctgcccctgggggcacagcacccttgcctccttggaagaccccttctagctcccagccaccacctcagccgcaggccaagcctcaggtccagctccatgtccagcctcaggccaagccccatgtccaaccccagcctgtgtcttctgctaatacacagccccggggtcccctttctcaggcaccaactccagcacctaagtttgctccagtggctcctaaatttactcccgtggtttccaagttcagccctggtgctccaagtggacctgggccacagcccaatcaaaaaatggtgcctccggatgctccttcttctgtgagcacaggctcccctcagccccctagcttcacctatgctcagcagaaggagaagcccctagttcaagagaagcagcacccacagcctccaccagctcaaaaccaaaaccaggtacgctctcctggaggcccaggccccttgaccctgaaggaggtagaggagttggagcagctgacccagcagctgatgcaggacatggaacaccctcagaggcagagcgtggcagtgaatgagtcctgtggcaaatgcaatcagccactggcccgtgcacagcctgcggttcgtgcactgggacaactgttccacatcacctgcttcacttgccatcagtgtcagcagcagctgcagggacagcagttctatagcctggagggagcaccatattgtgagggctgctacaccgacactttggagaagtgcaacacctgtgggcagcccatcactgaccgcatgctgagggccactggcaaagcctaccacccacagtgcttcacctgtgtggtctgcgcctgtcccctggagggcacctccttcattgtggaccaggccaatcagccccactgtgtccctgactatcacaagcaatacgctccaaggtgctccgtctgctcggagccaatcatgcctgagcctggccgagacgagactgtgcgagtagtggcgctggataagaactttcatatgaagtgttacaagtgtgaggactgtgggaaacctctgtccattgaggcagatgacaacggctgtttccctctggatggccacgtcctttgtcggaagtgccactccgctagagcccagacctga

SEQ ID NO: 8 - protein, 564 aa, Murine sp.
MAAPRPPPAISVSVSAPAFYAPQKKFAPVVAPKPKVNPFRPGDSEPPVAAGAQRAQMGRVGEIPPPPPEDFPLPPPPLIGEGDDSEGALGGAFPPPPPPMIEEPFPPAPLEEDIFPSPPPPLEEEGGPEAPTQLPPQPREKVCSIDLEIDSLSSLLDDMTKNDPFKARVSSGYVPPPVATPFVPKPSTKPAPGGTAPLPPWKTPSSSQPPPQPQAKPQVQLHVQPQAKPHVQPQPVSSANTQPRGPLSQAPTPAPKFAPVAPKFTPVVSKFSPGAPSGPGPQPNQKMVPPDAPSSVSTGSPQPPSFTYAQQKEKPLVQEKQHPQPPPAQNQNQVRSPGGPGPLTLKEVEELEQLTQQLMQDMEHPQRQSVAVNESCGKCNQPLARAQPAVRALGQLFHITCFTCHQCQQQLQGQQFYSLEGAPYCEGCYTDTLEKCNTCGQPITDRMLRATGKAYHPQCFTCVVCACPLEGTSFIVDQANQPHCVPDYHKQYAPRCSVCSEPIMPEPGRDETVRVVALDKNFHMKCYKCEDCGKPLSIEADDNGCFPLDGHVLCRKCHSARAQT

SEQ ID NO: 9 - DNA, 1626 bp, Gallus gallus
  CDS (1)..(1626)
atggcttctccaggtaccccagggacccgtatgacaaccacagtcagtatcaacatttccacaccgtccttttacaacccacagaagaaatttgcacccgtggttgcccctaaacccaaggtgaatcccttcaagactgggggtacatcggagtcatcgcagccacagcctcctggaactggtgcccagcgtgcccagatagggagagtgggagagatccccgtatctgtgacagcagaagagctgccgctgccacctcctcccccacctggagaggagctaagtttctcctcaaactgtgcttttcctccacccccaccaccctttgaagagcctttcccaccagccccagatgaagcttttccttctcctcctccacctcctccaccaatgtttgatgaaggacctgccctacagatacctccaggatccacgggttctgtggagaaaccgttggccccaaaagctcacgtggaaatctcatctgcacccagagatcctactcctccttttccttccaagttcactccaaagccaagtggtaccttatcttccaagccccctggattggattcaactcctgccccagctccatgggcagctccacagcagcgcaaggagcccctagcctcagtccctccacccccctctctcccttctcagcctactgctaaattcacaccaccccctgttgccagctctcctggatccaaaccaggtgccactgttcccatggctccttcaaactctacaagatatcctacatcccttcagactcagttcactgccccttcaccctccggtcccttgtctcgacctcagcctcccaatttcacctatgctcagcagtgggaaagacctcaggtgcaggagaaacctgttcccactgaaaaatctgctgctgtaaaagacatgcgtagacccactgcagatccgcctaagggaaactctcctctgaccatgaaggaggtagaagagctggagctgttgacccagaaactaatgaaggatatggatcatccacctccagtagaagctgctacttctgagctctgtggcttctgtcggaagcccctgtcacggacccagccagctgtgagagctctggactgccttttccacgtggagtgcttcacctgcttcaagtgtgagaagcagctgcaggggcagcagttctacaatgtggatgaaaagcccttctgcgaggactgctatgctggaaccctggaaaagtgcagtgtctgcaaacagactatcacagacaggatgctgaaggccaccggtaactcataccatcctcagtgcttcacctgtgtgatgtgccatactcctctggagggggcctccttcatagtggaccaggccaaccagcctcactgtgtggatgactaccacaggaagtatgctccacgctgctcagtatgtagtgaacctatcatgccagagcctgggaaagatgagacagtgcgtgtggtggcactggagaaaaacttccacatgaaatgttacaagtgtgaggactgtgggaggcccttatctattgaggctgatgaaaatggctgctttccactggatgggcacgtactatgtatgaaatgtcacactgttcgtgctaaaacagcgtgc

SEQ ID NO: 10 - protein, 542 aa, Gallus gallus
MASPGTPGTRMTTTVSINISTPSFYNPQKKFAPVVAPKPKVNPFKTGGTSESSQPQPPGTGAQRAQIGRVGEIPVSVTAEELPLPPPPPPGEELSFSSNCAFPPPPPPFEEPFPPAPDEAFPSPPPPPPPMFDEGPALQIPPGSTGSVEKPLAPKAHVEISSAPRDPTPPFPSKFTPKPSGTLSSKPPGLDSTPAPAPWAAPQQRKEPLASVPPPPSLPSQPTAKFTPPPVASSPGSKPGATVPMAPSNSTRYPTSLQTQFTAPSPSGPLSRPQPPNFTYAQQWERPQVQEKPVPTEKSAAVKDMRRPTADPPKGNSPLTMKEVEELELLTQKLMKDMDHPPPVEAATSELCGFCRKPLSRTQPAVRALDCLFHVECFTCFKCEKQLQGQQFYNVDEKPFCEDCYAGTLEKCSVCKQTITDRMLKATGNSYHPQCFTCVMCHTPLEGASFIVDQANQPHCVDDYHRKYAPRCSVCSEPIMPEPGKDETVRVVALEKNFHMKCYKCEDCGRPLSIEADENGCFPLDGHVLCMKCHTVRAKTAC

SEQ ID NO: 11 - DNA, 1716 bp, Homo sapiens
  CDS (1)..(1716)
atggcggccccccgcccgtctcccgcgatctccgtttcggtctcggctccggctttttacgccccgcagaagaagttcggccctgtggtggccccaaagcccaaagtgaatcccttccggcccggggacagcgagcctcccccggcacccggggcccagcgcgcacagatgggccgggtgggcgagattcccccgccgcccccggaagactttcccctgcctccacctccccttgctggggatggcgacgatgcagagggtgctctgggaggtgccttcccgccgccccctcccccgatcgaggaatcatttccccctgcgcctctggaggaggagatcttcccttccccgccgcctcctccggaggaggagggagggcctgaggcccccataccgcccccaccacagcccagggagaaggtgagcagtattgatttggagatcgactctctgtcctcactgctggatgacatgaccaagaatgatcctttcaaagcccgggtgtcatctggatatgtgcccccaccagtggccactccattcagttccaagtccagtaccaagcctgcagccgggggcacagcacccctgcctccttggaagtccccttccagctcccagcctctgccccaggttccggctccggctcagagccagacacagttccatgttcagccccagccccagcccaagcctcaggtccaactccatgtccagtcccagacccagcctgtgtctttggctaacacccagccccgagggcccccagcctcatctccggctccagcccctaagttttctccagtgactcctaagtttactcctgtggcttccaagttcagtcctggagccccaggtggatctgggtcacaaccaaatcaaaaattggggcaccccgaagctctttctgctggcacaggctcccctcaacctcccagcttcacctatgcccagcagagggagaagccccgagtgcaggagaagcagcaccccgtgcccccaccggctcagaaccaaaaccaggtgcgctcccctggggccccagggcccctgactctgaaggaggtggaggagctggagcagctgacccagcagctaatgcaggacatggagcatcctcagaggcagaatgtggctgtcaacgaactctgcggccgatgccatcaacccctggcccgggcgcagccagccgtccgcgctctagggcagctgttccacatcgcctgcttcacctgccaccagtgtgcgcagcagctccagggccagcagttctacagtctggagggggcgccgtactgcgagggctgttacactgacaccctggagaagtgtaacacctgcggggagcccatcactgaccgcatgctgagggccacgggcaaggcctatcacccgcactgcttcacctgtgtggtctgcgcccgccccctggagggcacctccttcatcgtggaccaggccaaccggccccactgtgtccccgactaccacaagcagtacgccccgaggtgctccgtctgctctgagcccatcatgcctgagcctggccgagatgagactgtgcgagtggtcgccctggacaagaacttccacatgaagtgttacaagtgtgaggactgcgggaagcccctgtcgattgaggcagatgacaatggctgcttccccctggacggtcacgtgctctgtcggaagtgccacactgctagagcccagacc

SEQ ID NO: 12 - protein, 572 aa, Homo sapiens
MAAPRPSPAISVSVSAPAFYAPQKKFGPVVAPKPKVNPFRPGDSEPPPAPGAQRAQMGRVGEIPPPPPEDFPLPPPPLAGDGDDAEGALGGAFPPPPPPIEESFPPAPLEEEIFPSPPPPPEEEGGPEAPIPPPPQPREKVSSIDLEIDSLSSLLDDMTKNDPFKARVSSGYVPPPVATPFSSKSSTKPAAGGTAPLPPWKSPSSSQPLPQVPAPAQSQTQFHVQPQPQPKPQVQLHVQSQTQPVSLANTQPRGPPASSPAPAPKFSPVTPKFTPVASKFSPGAPGGSGSQPNQKLGHPEALSAGTGSPQPPSFTYAQQREKPRVQEKQHPVPPPAQNQNQVRSPGAPGPLTLKEVEELEQLTQQLMQDMEHPQRQNVAVNELCGRCHQPLARAQPAVRALGQLFHIACFTCHQCAQQLQGQQFYSLEGAPYCEGCYTDTLEKCNTCGEPITDRMLRATGKAYHPHCFTCVVCARPLEGTSFIVDQANRPHCVPDYHKQYAPRCSVCSEPIMPEPGRDETVRVVALDKNFHMKCYKCEDCGKPLSIEADDNGCFPLDGHVLCRKCHTARAQT

SEQ ID NO: 13 - DNA, 1836 bp, Homo sapiens
  CDS (1)..(1836)
atgtctcacccatcttggctgccacccaaaagcactggtgagcccctcggccatgtgcctgcacggatggagaccacccattcctttgggaaccccagcatttcagtgtctacacaacagccacccaaaaagtttgccccggtagttgctccaaaacctaagtacaacccatacaaacaacctggaggtgagggtgattttcttccacccccacctccacctctagatgattccagtgcccttccatctatctctggaaactttcctcctccaccacctcttgatgaagaggctttcaaagtacaggggaatcccggaggcaagacacttgaggagaggcgctccagcctggacgctgagattgactccttgaccagcatcttggctgaccttgagtgcagctccccctataagcctcggcctccacagagctccactggttcaacagcctctcctccagtttcgaccccagtcacaggacacaagagaatggtcatcccgaaccaaccccctctaacagcaaccaagaagtctacattgaaaccacagcctgcaccccaggctggacccatccctgtggctccaatcggaacactcaaaccccagcctcagccagtcccagcctcctacaccacggcctccacttcttcaaggcctacctttaatgtgcaggtgaagtcagcccagcccagccctcattatatggctgccccttcatcaggacaaatttatggctcagggccccagggctataacactcagccagttcctgtctctgggcagtgtccacctccttcaacacggggaggcatggattatgcctacattccaccaccaggacttcagccggagcctgggtatgggtatgcccccaaccagggacgctattatgaaggctactatgcagcagggccaggctatgggggcagaaatgactctgaccctacctatggtcaacaaggtcacccaaatacctggaaacgggaaccagggtacactcctcctggagcagggaaccagaaccctcctgggatgtatccagtcactggtcccaagaagacctatatcacagatcctgtttcagccccctgtgcgccaccattgcagccaaagggtggccattcagggcaactggggccttcgtcagttgccccttcattccgcccagaggatgagcttgagcacctgaccaaaaagatgctgtatgacatggaaaatccacctgctgacgaatactttggccgctgtgctcgctgtggagaaaacgtagttggggaaggtacaggatgcactgccatggatcaggtcttccacgtggattgttttacctgcatcatctgcaacaacaagctccgagggcagccattctatgctgtggaaaagaaagcatactgcgagccctgctacattaatactctggagcagtgcaatgtgtgttccaagcccatcatggagcggattctccgagccaccgggaaggcctatcatcctcactgtttcacctgcgtgatgtgccaccgcagcctggatgggatcccattcactgtggatgctggcgggctcattcactgcattgaggacttccacaagaaatttgccccgcggtgttctgtgtgcaaggagcctattatgccagccccgggccaggaggagactgtccgtattgtggctttggatcgagatttccatgttcactgctaccgatgcgaggattgcggtggtctcctgtctgaaggagataaccaaggctgctaccccttggatgggcacatcctctgcaagacctgcaactctgcccgcatcagggtgttgaccgccaaggcgagcactgacctt

SEQ ID NO: 14 - protein, 612 aa, Homo sapiens
MSHPSWLPPKSTGEPLGHVPARMETTHSFGNPSISVSTQQPPKKFAPVVAPKPKYNPYKQPGGEGDFLPPPPPPLDDSSALPSISGNFPPPPPLDEEAFKVQGNPGGKTLEERRSSLDAEIDSLTSILADLECSSPYKPRPPQSSTGSTASPPVSTPVTGHKRMVIPNQPPLTATKKSTLKPQPAPQAGPIPVAPIGTLKPQPQPVPASYTTASTSSRPTFNVQVKSAQPSPHYMAAPSSGQIYGSGPQGYNTQPVPVSGQCPPPSTRGGMDYAYIPPPGLQPEPGYGYAPNQGRYYEGYYAAGPGYGGRNDSDPTYGQQGHPNTWKREPGYTPPGAGNQNPPGMYPVTGPKKTYITDPVSAPCAPPLQPKGGHSGQLGPSSVAPSFRPEDELEHLTKKMLYDMENPPADEYFGRCARCGENVVGEGTGCTAMDQVFHVDCFTCIICNNKLRGQPFYAVEKKAYCEPCYINTLEQCNVCSKPIMERILRATGKAYHPHCFTCVMCHRSLDGIPFTVDAGGLIHCIEDFHKKFAPRCSVCKEPIMPAPGQEETVRIVALDRDFHVHCYRCEDCGGLLSEGDNQGCYPLDGHILCKTCNSARIRVLTAKASTDL

SEQ ID NO: 15 - DNA, 1431 bp, Homo sapiens
  CDS (1)..(1428)
atgtcggggcccacctggctgcccccgaagcagccggagcccgccagagcccctcaggggagggcgatcccccgcggcaccccggggccaccaccggcccacggagcagcactccagccccaccccagggtcaatttttgcccccttccatctgagcagtgttaccaggccccagggggaccggaggatcgggggccggcgtgggtggggtcccatggagtactccagcacacgcaggggctccctgcagacagggggggccttcgccctggaagcctggacgccgagatagacttgctgagcagcacgctggccgagctgaatgggggtcggggtcatgcgtcacggcgaccagaccgacaggcatatgagcccccgccacctcctgcctaccgcacgggctgcctgaagccaaatccagcctcgccgctcccagcgtctccctatgggggccccactccagcctcttacactaccgccagcaccccggctggcccagccttccccgtgcaagtgaaggtggcacagccagtgaggggctgcggcccacccaggcggggagcctctcaggcctctgggcccctcccgggcccccactttcctctcccaggccgaggtgaagtctgggggcctggctataggagccagagagagccagggccaggggccaaagaggaagctgctggggtctctggccctgcaggaagaggaagaggaggcgagcacgggccccaggtgcccctgagccagcctccagaggatgagctggataggctgacgaagaagctggttcacgacatgaaccacccgcccagcggggagtactttggccagtgtggtggctgcggagaagatgtggttggggatggggctggggttgtggcccttgatcgcgtctttcacgtgggctgctttgtatgttctacatgccgggcccagcttcgcggccagcatttctacgccgtggagaggagggcatattgcgagggctgctacgtggccaccctggagaaatgtgccacgtgctcccagcccatcctggaccggatcctgcgggctatggggaaggcctaccaccctggctgcttcacctgcgtggtgtgtcaccgcggcctcgacggcatccccttcacagtggatgctacgagccagatccactgcattgaggactttcacaggaagtttgccccaagatgctcagtgtgcggtggggccataatgcctgagccaggtcaggaggagactgtgagaattgttgctctggatcgaagttttcacattggctgttacaagtgcgaggagtgtgggctgctgctctcctctgagggcgagtgtcagggctgctacccgctggatgggcacatcttgtgcaaggcctgcagcgcctggcgcatccaggagctctcagccaccgtcaccactgactgctga

SEQ ID NO: 16 - protein, 476 aa, Homo sapiens [sequence omitted]

SEQ ID NO: 17 - DNA, 1443 bp, Murine sp.
  CDS (1)..(1440)
atgtccgggcccacctggcttcccccgaagcagccagaaccctccagactccctcaggggagatcgctgcccagaggcgccctgggcccgccaacggcccacggagcaacactccagcctcaccccagggtcaacttttgccccctcccgcctgaacactgttatcagcctccgggggtaccggaagatcgggggcctacttgggtgggatcccatggaacaccccagcgcctgcaggggctccctccagacagggggatcatccgccctggcagtctggatgctgagatagattcgctcaccagcatgttggctgatctggacgggggtcgcagtcatgcacctaggcggccagacagacaggcttttgaggctcccccaccccatgcttaccgcggaggctccctgaagcccagtggaggtgctgttccaaccccgatgctcccagcatcccactatggtggacctaccccagcctcctatgctaccgcgagcacgccagctggccctgctttccctgtacaagtgaaggtggctcaacctgtgagaggctgtggactgcccaggcgaggggcctctcaggcctctgggcctcttccaggcccccactttcctctgacaggtcgtggtgaagtctggggggctggctataggagccaccgagagccaggaccgggggttccggagggaccttctggagtacatatccctgcaggaggagggagaggaggtgggcatgagcctcagggccccttaggccaacctcctgaagaggaactggagagactgaccaagaaactggtgcatgacatgagccaccctcccagtggggagtactttggtcggtgtggtggctgtggcgaagatgtggtgggcgatggagctggggttgtggccctggaccgtgtcttccatattggttgctttgtgtgttctacctgtcgggcccagctccggggccagcacttctatgctgtggagaggcgggcatattgtgagagctgctatgtggccaccctggagaaatgttccacatgctctgaacccatcctggaccgaatcctgagggctatggggaaggcgtaccaccctggttgcttcacctgtgtggtatgccaccgtggtcttgatggcatcccgttcacagtggacgccaccagccagatccactgcattgaagatttccacaggaaatttgccccacgatgctcagtgtgtggtggggccatcatgccggaaccaggtcaggaggagacggtgagaatcgttgctctggatcgaagtttccacatcggctgttacaagtgtgaggagtgtgggctgctgctgtcctctgagggagagtgtcaaggctgctacccgctggatgggcacatcttgtgcaaggcttgcagcgcctggcgtatccaagagctctcagccactgtcaccactgattgttga

SEQ ID NO: 18 - protein, 480 aa, Murine sp.
MSGPTWLPPKQPEPSRLPQGRSLPRGALGPPTAHGATLQPHPRVNFCPLPPEHCYQPPGVPEDRGPTWVGSHGTPQRLQGLPPDRGIIRPGSLDAEIDSLTSMLADLDGGRSHAPRRPDRQAFEAPPPHAYRGGSLKPSGGAVPTPMLPASHYGGPTPASYATASTPAGPAFPVQVKVAQPVRGCGLPRRGASQASGPLPGPHFPLTGRGEVWGAGYRSHREPGPGVPEGPSGVHIPAGGGRGGGHEPQGPLGQPPEEELERLTKKLVHDMSHPPSGEYFGRCGGCGEDVVGDGAGVVALDRVFHIGCFVCSTCRAQLRGQHFYAVERRAYCESCYVATLEKCSTCSEPILDRILRAMGKAYHPGCFTCVVCHRGLDGIPFTVDATSQIHCIEDFHRKFAPRCSVCGGAIMPEPGQEETVRIVALDRSFHIGCYKCEECGLLLSSEGECQGCYPLDGHILCKACSAWRIQELSATVTTDC

SEQ ID NO: 19 - DNA, 1122 bp, Murine sp.
  CDS (1)..(1122)
gcggccccccgcccgcctcccgcgatctccgtctccgtctcggcccccgcgttttacgccccgcagaagaagttcgccccggttgtggccccaaagcccaaagtgaatcctttccggcctggggacagcgagcctcctgtagcagccggggcccaaagagcgcagatgggtcgggtgggcgagatcccaccaccacccccggaagactttcctttgccccctcctccccttattggggagggcgacgactcagagggtgccctgggaggtgccttcccacctccacctcccccgatgatcgaggaaccattcccccctgctcctctggaggaggacatcttcccctcccctccacctccactggaggaggagggagggcctgaggcccctacccagctcccaccgcagcccagggagaaagtgtgcagtattgacctggagattgactctctgtcctcactgctggacgacatgaccaagaacgatcccttcaaagcccgggtatcatccggatatgtacccccaccagttgccactccatttgttcccaagcctagtaccaaacctgcccctgggggcacagcacccttgcctccttggaagaccccttctagctcccagccaccacctcagccgcaggccaagcctcaggtccagctccatgtccagcctcaggccaagccccatgtccaaccccagcctgtgtcttctgctaatacacagccccggggtcccctttctcaggcaccaactccagcacctaagtttgctccagtggctcctaaatttactcccgtggtttccaagttcagccctggtgctccaagtggacctgggccacagcccaatcaaaaaatggtgcctccggatgctccttcttctgtgagcacaggctcccctcagccccctagcttcacctatgctcagcagaaggagaagcccctagttcaagagaagcagcacccacagcctccaccagctcaaaaccaaaaccaggtacgctctcctggaggcccaggccccttgaccctgaaggaggtagaggagttggagcagctgacccagcagctgatgcaggacatggaacaccctcagaggcagagcgtggcagtgaatgagtcc

SEQ ID NO: 20 - protein, 374 aa, Murine sp.
AAPRPPPAISVSVSAPAFYAPQKKFAPVVAPKPKVNPFRPGDSEPPVAAGAQRAQMGRVGEIPPPPPEDFPLPPPPLIGEGDDSEGALGGAFPPPPPPMIEEPFPPAPLEEDIFPSPPPPLEEEGGPEAPTQLPPQPREKVCSIDLEIDSLSSLLDDMTKNDPFKARVSSGYVPPPVATPFVPKPSTKPAPGGTAPLPPWKTPSSSQPPPQPQAKPQVQLHVQPQAKPHVQPQPVSSANTQPRGPLSQAPTPAPKFAPVAPKFTPVVSKFSPGAPSGPGPQPNQKMVPPDAPSSVSTGSPQPPSFTYAQQKEKPLVQEKQHPQPPPAQNQNQVRSPGGPGPLTLKEVEELEQLTQQLMQDMEHPQRQSVAVNES

SEQ ID NO: 21 - DNA, 1053 bp, Gallus gallus
  CDS (1)..(1053)
atggcttctccaggtaccccagggacccgtatgacaaccacagtcagtatcaacatttccacaccgtccttttacaacccacagaagaaatttgcacccgtggttgcccctaaacccaaggtgaatcccttcaagactgggggtacatcggagtcatcgcagccacagcctcctggaactggtgcccagcgtgcccagatagggagagtgggagagatccccgtatctgtgacagcagaagagctgccgctgccacctcctcccccacctggagaggagctaagtttctcctcaaactgtgcttttcctccacccccaccaccctttgaagagcctttcccaccagccccagatgaagcttttccttctcctcctccacctcctccaccaatgtttgatgaaggacctgccctacagatacctccaggatccacgggttctgtggagaaaccgttggccccaaaagctcacgtggaaatctcatctgcacccagagatcctactcctccttttccttccaagttcactccaaagccaagtggtaccttatcttccaagccccctggattggattcaactcctgccccagctccatgggcagctccacagcagcgcaaggagcccctagcctcagtccctccacccccctctctcccttctcagcctactgctaaattcacaccaccccctgttgccagctctcctggatccaaaccaggtgccactgttcccatggctccttcaaactctacaagatatcctacatcccttcagactcagttcactgccccttcaccctccggtcccttgtctcgacctcagcctcccaatttcacctatgctcagcagtgggaaagacctcaggtgcaggagaaacctgttcccactgaaaaatctgctgctgtaaaagacatgcgtagacccactgcagatccgcctaagggaaactctcctctgaccatgaaggaggtagaagagctggagctgttgacccagaaactaatgaaggatatggatcatccacctccagtagaagctgctacttctgagctc

SEQ ID NO: 22 - protein, 351 aa, Gallus gallus
MASPGTPGTRMTTTVSINISTPSFYNPQKKFAPVVAPKPKVNPFKTGGTSESSQPQPPGTGAQRAQIGRVGEIPVSVTAEELPLPPPPPPGEELSFSSNCAFPPPPPPFEEPFPPAPDEAFPSPPPPPPPMFDEGPALQIPPGSTGSVEKPLAPKAHVEISSAPRDPTPPFPSKFTPKPSGTLSSKPPGLDSTPAPAPWAAPQQRKEPLASVPPPPSLPSQPTAKFTPPPVASSPGSKPGATVPMAPSNSTRYPTSLQTQFTAPSPSGPLSRPQPPNFTYAQQWERPQVQEKPVPTEKSAAVKDMRRPTADPPKGNSPLTMKEVEELELLTQKLMKDMDHPPPVEAATSEL

SEQ ID NO: 23 - DNA, 1140 bp, Homo sapiens
  CDS (1)..(1140)
atggcggccccccgcccgtctcccgcgatctccgtttcggtctcggctccggctttttacgccccgcagaagaagttcggccctgtggtggccccaaagcccaaagtgaatcccttccggcccggggacagcgagcctcccccggcacccggggcccagcgcgcacagatgggccgggtgggcgagattcccccgccgcccccggaagactttcccctgcctccacctccccttgctggggatggcgacgatgcagagggtgctctgggaggtgccttcccgccgccccctcccccgatcgaggaatcatttccccctgcgcctctggaggaggagatcttcccttccccgccgcctcctccggaggaggagggagggcctgaggcccccataccgcccccaccacagcccagggagaaggtgagcagtattgatttggagatcgactctctgtcctcactgctggatgacatgaccaagaatgatcctttcaaagcccgggtgtcatctggatatgtgcccccaccagtggccactccattcagttccaagtccagtaccaagcctgcagccgggggcacagcacccctgcctccttggaagtccccttccagctcccagcctctgccccaggttccggctccggctcagagccagacacagttccatgttcagccccagccccagcccaagcctcaggtccaactccatgtccagtcccagacccagcctgtgtctttggctaacacccagccccgagggcccccagcctcatctccggctccagcccctaagttttctccagtgactcctaagtttactcctgtggcttccaagttcagtcctggagccccaggtggatctgggtcacaaccaaatcaaaaattggggcaccccgaagctctttctgctggcacaggctcccctcaacctcccagcttcacctatgcccagcagagggagaagccccgagtgcaggagaagcagcaccccgtgcccccaccggctcagaaccaaaaccaggtgcgctcccctggggccccagggcccctgactctgaaggaggtggaggagctggagcagctgacccagcagctaatgcaggacatggagcatcctcagaggcagaatgtggctgtc

SEQ ID NO: 24 - protein, 380 aa, Homo sapiens
MAAPRPSPAISVSVSAPAFYAPQKKFGPVVAPKPKVNPFRPGDSEPPPAPGAQRAQMGRVGEIPPPPPEDFPLPPPPLAGDGDDAEGALGGAFPPPPPPIEESFPPAPLEEEIFPSPPPPPEEEGGPEAPIPPPPQPREKVSSIDLEIDSLSSLLDDMTKNDPFKARVSSGYVPPPVATPFSSKSSTKPAAGGTAPLPPWKSPSSSQPLPQVPAPAQSQTQFHVQPQPQPKPQVQLHVQSQTQPVSLANTQPRGPPASSPAPAPKFSPVTPKFTPVASKFSPGAPGGSGSQPNQKLGHPEALSAGTGSPQPPSFTYAQQREKPRVQEKQHPVPPPAQNQNQVRSPGAPGPLTLKEVEELEQLTQQLMQDMEHPQRQNVAV

SEQ ID NO: 25 - DNA, 1236 bp, Homo sapiens
  CDS (1)..(1236)
ccatcttggctgccacccaaaagcactggtgagcccctcggccatgtgcctgcacggatggagaccacccattcctttgggaaccccagcatttcagtgtctacacaacagccacccaaaaagtttgccccggtagttgctccaaaacctaagtacaacccatacaaacaacctggaggtgagggtgattttcttccacccccacctccacctctagatgattccagtgcccttccatctatctctggaaactttcctcctccaccacctcttgatgaagaggctttcaaagtacaggggaatcccggaggcaagacacttgaggagaggcgctccagcctggacgctgagattgactccttgaccagcatcttggctgaccttgagtgcagctccccctataagcctcggcctccacagagctccactggttcaacagcctctcctccagtttcgaccccagtcacaggacacaagagaatggtcatcccgaaccaaccccctctaacagcaaccaagaagtctacattgaaaccacagcctgcaccccaggctggacccatccctgtggctccaatcggaacactcaaaccccagcctcagccagtcccagcctcctacaccacggcctccacttcttcaaggcctacctttaatgtgcaggtgaagtcagcccagcccagccctcattatatggctgccccttcatcaggacaaatttatggctcagggccccagggctataacactcagccagttcctgtctctgggcagtgtccacctccttcaacacggggaggcatggattatgcctacattccaccaccaggacttcagccggagcctgggtatgggtatgcccccaaccagggacgctattatgaaggctactatgcagcagggccaggctatgggggcagaaatgactctgaccctacctatggtcaacaaggtcacccaaatacctggaaacgggaaccagggtacactcctcctggagcagggaaccagaaccctcctgggatgtatccagtcactggtcccaagaagacctatatcacagatcctgtttcagccccctgtgcgccaccattgcagccaaagggtggccattcagggcaactggggccttcgtcagttgccccttcattccgcccagaggatgagcttgagcacctgaccaaaaagatgctgtatgacatggaaaatccacctgctgacgaatactttggccgc

SEQ ID NO: 26 - protein, 412 aa, Homo sapiens
PSWLPPKSTGEPLGHVPARMETTHSFGNPSISVSTQQPPKKFAPVVAPKPKYNPYKQPGGEGDFLPPPPPPLDDSSALPSISGNFPPPPPLDEEAFKVQGNPGGKTLEERRSSLDAEIDSLTSILADLECSSPYKPRPPQSSTGSTASPPVSTPVTGHKRMVIPNQPPLTATKKSTLKPQPAPQAGPIPVAPIGTLKPQPQPVPASYTTASTSSRPTFNVQVKSAQPSPHYMAAPSSGQIYGSGPQGYNTQPVPVSGQCPPPSTRGGMDYAYIPPPGLQPEPGYGYAPNQGRYYEGYYAAGPGYGGRNDSDPTYGQQGHPNTWKREPGYTPPGAGNQNPPGMYPVTGPKKTYITDPVSAPCAPPLQPKGGHSGQLGPSSVAPSFRPEDELEHLTKKMLYDMENPPADEYFGR

SEQ ID NO: 27 - DNA, 3201 bp, Homo sapiens
  CDS (1)..(3198)
atgccagtgtttcatacgcgcacgatcgagagcatcctggagccggtggcacagcagatctcccacctggtgataatgcacgaggagggcgaggtggacggcaaagccattcctgacctcaccgcgcccgtggccgccgtgcaggcggccgtcagcaacctcgtccgggttggaaaagagactgttcaaaccactgaggatcagattttgaagagagatatgccaccagcatttattaaggttgagaatgcttgcaccaagcttgtccaggcagctcagatgcttcagtcagacccttactcagtgcctgctcgagattatctaattgatgggtcaaggggcatcctctctggaacatcagacctgctccttaccttcgatgaggctgaggtccgtaaaattattagagtttgcaaaggaattttggaatatcttacagtggcagaggtggtggagactatggaagatttggtcacttacacaaagaatcttgggccaggaatgactaagatggccaagatgattgacgagagacagcaggagctcactcaccaggagcaccgagtgatgttggtgaactcgatgaacaccgtgaaagagttgctgccagttctcatttcagctatgaagatttttgtaacaactaaaaactcaaaaaaccaaggcatagaggaagctttaaaaaatcgcaattttactgtagaaaaaatgagtgctgaaattaatgagataattcgtgtgttacaactcacctcttgggatgaagatgcctgggccagcaaggacactgaagccatgaagagagcattggcctccatagactccaaactgaaccaggccaaaggttggctccgtgaccctagtgcctccccaggggatgctggtgagcaggccatcagacagatcttagatgaagctggaaaagttggtgaactctgtgcaggcaaagaacgcagggagattctgggaacttgcaaaatgctagggcagatgactgatcaagtggctgacctccgtgccagaggacaaggatcctcaccggtggccatgcagaaagctcagcaggtatctcagggtctggatgtgctcacagcaaaagtggaaaatgcagctcgcaagctggaagccatgaccaactcaaagcagagcattgcaaagaagatcgatgctgctcagaactggcttgcagatccaaatggtggaccggaaggagaagagcagattcgaggtgctttggctgaagctcggaaaatagcagaattatgtgatgatcctaaagaaagagatgacattctacgttcccttggggaaatatctgctctgacttctaaattagcagatctacgaagacaggggaaaggagattctccagaggctcgagccttggccaaacaggtggccacggccctgcagaacctgcagaccaaaaccaaccgggctgtggccaacagcagaccggccaaagcagctgtacaccttgagggcaagattgagcaagcacagcggtggattgataatcccacagtggatgaccgtggagtcggtcaggctgccatccgggggcttgtggccgaagggcatcgtctggctaatgttatgatggggccttatcggcaagatcttctcgccaagtgtgaccgagtggaccagctgacagcccagctggctgacctggctgccagaggggaaggggagagtcctcaggcacgagcacttgcatctcagctccaagactccttaaaggatctaaaagctcggatgcaggaggccatgactcaggaagtgtcagatgttttcagcgataccacaactcccatcaagctgttggcagtggcagccacggcgcctcctgatgcgcctaacagggaagaggtatttgatgagagggcagctaactttgaaaaccattcaggaaagcttggtgctacggccgagaaggcggctgcggttggtactgctaataaatcaacagtggaaggcattcaggcctcagtgaagacggcccgagaactcacaccccaggtggtctcggctgctcgtatcttacttaggaaccctggaaatcaagctgcttatgaacattttgagaccatgaagaaccagtggatcgataatgttgaaaaaatgacagggctggtggacgaagccattgataccaaatctctgttggatgcttcagaagaagcaattaaaaaagacctggacaagtgcaaggtagctatggccaacattcagcctcagatgctggttgctggggcaaccagtattgctcgtcgggccaaccggatcctgctggtggctaagagggaggtggagaattccgaggatcccaagttccgtgaggctgtgaaagctgcctctgatgaattgagcaaaaccatctccccaatggtgatggatgcaaaagctgtggctggaaacatttccgaccctggactgcaaaagagcttcctggactcaggatatcggatcctgggagctgtggccaaggtcagagaagccttccaacctcaggagcctgacttcccgccgcctccaccagaccttgaacaactccgactaacagatgagcttgctcctcccaaaccacctctgcctgaaggtgaggtccctccacctaggcctccaccaccagaggaaaaggatgaagagttccctgagcagaaggccggggaggtgattaaccagccaatgatgatggctgccagacagctccatgatgaagctcgcaaatggtccagcaagggcaatgacatcattgcagcagccaagcgcatggctctgctgatggctgagatgtctcggctggtaagagggggcagtggtaccaagcgggcactcattcagtgtgccaaggacatcgccaaggcctcagatgaggtgactcggttggccaaggaggttgccaagcagtgcacagataaacggattagaaccaacctcttacaggtatgtgagcgaatcccaaccataagcacccagctcaaaatcctgtccacagtgaaggccaccatgctgggccggaccaacatcagtgatgaggagtctgagcaggccacagagatgctggttcacaatgcccagaacctcatgcagtctgtgaaggagactgtgcgggaagctgaagctgcttcaatcaaaattcgaacagatgctggatttacactgcgctgggttagaaagactccctggtaccagtag

SEQ ID NO: 28 - protein, 1066 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 29 - DNA, 681 bp, Homo sapiens
  CDS (1)..(681)
cctgacttcccgccgcctccaccagaccttgaacaactccgactaacagatgagcttgctcctcccaaaccacctctgcctgaaggtgaggtccctccacctaggcctccaccaccagaggaaaaggatgaagagttccctgagcagaaggccggggaggtgattaaccagccaatgatgatggctgccagacagctccatgatgaagctcgcaaatggtccagcaagggcaatgacatcattgcagcagccaagcgcatggctctgctgatggctgagatgtctcggctggtaagagggggcagtggtaccaagcgggcactcattcagtgtgccaaggacatcgccaaggcctcagatgaggtgactcggttggccaaggaggttgccaagcagtgcacagataaacggattagaaccaacctcttacaggtatgtgagcgaatcccaaccataagcacccagctcaaaatcctgtccacagtgaaggccaccatgctgggccggaccaacatcagtgatgaggagtctgagcaggccacagagatgctggttcacaatgcccagaacctcatgcagtctgtgaaggagactgtgcgggaagctgaagctgcttcaatcaaaattcgaacagatgctggatttacactgcgctgggttagaaagactccctggtaccag

SEQ ID NO: 30 - protein, 227 aa, Homo sapiens
PDFPPPPPDLEQLRLTDELAPPKPPLPEGEVPPPRPPPPEEKDEEFPEQKAGEVINQPMMMAARQLHDEARKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 31 - protein, 502 aa, Homo sapiens
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 32 - protein, 505 aa, Homo sapiens
MSSVQQQPPPPRRVTNVGSLLLTPQENESLFTFLGKKCVTMSSAVVQLYAADRNCMWSKKCSGVACLVKDNPQRSHFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKGDIGTPSNFQHIGHVGWDPNTGSDLNNLDPELKNLFDMCGILEAQLKERETLKVIYDFIEKTGGVEAVKNELRRQAPPPPPPSRGGPPPPPPPPHSSGPPPPPARGRGAPPPPPSRAPTAAPPPPPPSRPSVEVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 33 - protein, 559 aa, Homo sapiens
MPLVKRNIDPRHLCHTALPRGIKNELECVTNISLANIIRQLSSLSKYAEDIFGELFNEAHSFSFRVNSLQERVDRLSVSVTQLDPKEEELSLQDITMRKAFRSSTIQDQQLFDRKTLPIPLQETYDVCEQPPPLNILTPYRDDGKEGLKFYTNPSYFFDLWKEKMLQDTEDKRKEKRKQKQKNLDRPHEPEKVPRAPHDRRREWQKLAQGPELAEDDANLLHKHIEVANGPASHFETRPQTYVDHMDGSYSLSALPFSQMSELLTRAEERVLVRPHEPPPPPPMHGAGDAKPIPTCISSATGLIENRPQSPATGRTPVFVSPTPPPPPPPLPSALSTSSLRASMTSTPPPPVPPPPPPPATALQAPAVPPPPAPLQIAPGVLHPAPPPIAPPLVQPSPPVARAAPVCETVPVHPLPQGEVQGLPPPPPPPPLPPPGIRPSSPVTVTALAHPPSGLHPTPSTAPGPHVPLMPPSPPSQVIPASEPKRHPSTLPVISDARSVLLEAIRKGIQLRKVEEQREQEAKHERIENDVATILSRRIAVEYSDSEDDSEFDEVDWLE

SEQ ID NO: 34 - protein, 520 aa, Murine sp.
MNSGPGPVGGRPGGRGGPAVQQNIPSNLLQDHENQRLFELLGRKCWTLATTVVQLYLALPPGAEHWTMEHCGAVCFVKDNPQKSYFIRLYALQAGRLLWEQELYSQLVYLTPTPFFHTFAGDDCQVGLNFADESEAQAFRALVQEKIQKRNQRQSGERRQLPPPPAPINEERRGGLPPVPPHPGGDHGGPSGGPLSLGLVTVDIQNPDITSSRYRGLPAPGPGPTDKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPCRGGGGGGGGGGGGGGGGGGQPLRPPVLGSNKGRSPPLPPVPMGGAPPPPTPRGPPPPGRGGPPPPPPPATGRSGPPPPPLPGAGGPPAPPPPPPPPPPPPCPGSGPAPPPLPPTPVSGGSPAPGGGRGALLDQIRQGIQLNKTPGALENSVQQPPAQQSEGLVGALMHVMQKRSRVIHSSDEGEDQTGEDEEDDEWDD

SEQ ID NO: 35 - protein, 501 aa, Rattus rattus
MSSGQQPPRRVTNVGSLLLTPQENESLFSFLGKKCVTMSSAVVQLYAADRNCMWSKKCSGVACLVKDNPQRSYFLRIFDIKDGKLLWEQELYNNFVYNSPRGYFHTFAGDTCQVALNFANEEEAKKFRKAVTDLLGRRQRKSEKRRDAPNGPNLPMATVDIKNPEITTNRFYSSQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHSSGPPPPPARGRGAPPPPPSRAPTAAPPPPPPSRPGVVVPPPPPNRMYPPPPPALPSSAPSGPPPPPPLSMAGSTAPPPPPPPPPPPGPPPPPGLPSDGDHQVPASSGNKAALLDQIREGAQLKKVEQNSRPVSCSGRDALLDQIRQGIQLKSVSDGQESTPPTPAPTSGIVGALMEVMQKRSKAIHSSDEDEDDDDEEDFQDDDEWED

SEQ ID NO: 36 - protein, 505 aa, Bos taurus
MSSGQQQPPPPRRVTNVGSLLLTPQENESLFTFLGKKCVTMSSAVVQLYAADRNCMWSKKCSGVACLVKDNPQRSYFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQINNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHSSGPPPPPARGRGAPPPPPSRAPTAAPPPPPPSRPGVGAPPPPPNRMYPPPLPALPSSAPSGPPPPPPPLSVSGSVAPPPPPPPPPPPGPPPPPGLPSDGDHQVPTPAGSKAALLDQIREGAQLKKVEQNSRPVSCSGRDALLDQIRQGIQLKSVTDAPESTPPAPAPTSGIVGALMEVMQKRSKAIHSSDEDEDEDDDEDFEDDDEWED

SEQ ID NO: 37 - protein, 633 aa, Saccharomyces cerevisiae
MGLLNSSDKEIIKRALPKASNKIIDVTVARLYIAYPDKNEWQYTGLSGALALVDDLVGNTFFLKLVDINGHRGVIWDQELYVNFEYYQDRTFFHTFEMEECFAGLLFVDINEASHFLKRVQKRERYANRKTLLNKNAVALTKKVREEQKSQVVHGPRGESLIDNQRKRYNYEDVDTIPTTKHKAPPPPPPTAETFDSDQTSSFSDINSTTASAPTTPAPALPPASPEVRKEETHPKHSLPPLPNQFAPLPDPPQHNSPPQNNAPSQPQSNPFPFPIPEIPSTQSATNPFPFPVPQQQFNQAPSMGIPQQNRPLPQLPNRNNRPVPPPPPMRTTTEGSGVRLPAPPPPPRRGPAPPPPPHRHVTSNTLNSAGGNSLLPQATGRRGPAPPPPPRASRPTPNVTMQQNPQQYNNSNRPFGYQTNSNMSSPPPPPVTTFNTLTPQMTAATGQPAVPLPQNTQAPSQATNVPVAPPPPPASLGQSQIPQSAPSAPIPPTLPSTTSAAPPPPPAFLTQQPQSGGAPAPPPPPQMPATSTSGGGSFAETTGDAGRDALLASIRGAGGIGALRKVDKSQLDKPSVLLQEARGESASPPAAAGNGGTPGGPPASLADALAAALNKRKTKVGAHDDMDNGDDW

SEQ ID NO: 38 - protein, 574 aa, Schizosaccharomyces pombe
MPPSSSITQEDKATIRKYIPKSTNKIIAAAVVKLYVAYPDPNKWNYTGLCGALVLSYDTTAKCCWFKLVDVVNNSGIIWDQELYQNMDYRQDRTFFHSFELDKCLAGFSFANETDAQKFYKKVLDKGCHPESIENPVLSFITRKGSSRHAPNNSNIQPPSAAPPVPGKENYNAVGSKSPNEPELLNSLDPSLIDSLMKMGISQDQIAENADFVKAYLNESAGTPTSTSAPPIPPSIPSSRPPERVPSVSAPAPPPIPPPSNGTVSSPPNSPPRPIAPVSMNPAINSTSKPPLPPPSSRVSAAALAANKKRPPPPPPPSRRNRGKPPIGNGSSNSSLPPPPPPPRSNAAGSIPLPPQGRSAPPPPPPRSAPSTGRQPPPLSSSRAVSNPPAPPPAIPGRSAPALPPLGNASRTSTPPVPTPPSLPPSAPPSLPPSAPPSLPMGAPAAPPLPPSAPIAPPLPAGMPAAPPLPPAAPAPPPAPAPAPAAPVASIAELPQQDGRANLMASIRASGGMDLLKSRKVSASPSVASTKTSNPPVEAPPSNNLMDALASALNQRKTKVAQSDEEDEDDDEWD

(19) United States
(12) Patent Application Publication
(10) Pub. No.: US 2003/0170726 A1
(43) Pub. Date: Sep. 11, 2003

(54) Peptides sequences comprising one or several protein binding units of the ena/vasp family and the uses thereof

(76) Inventors: Julie Fradelizi, Paris (FR); Evelyne Friederich, Luxembourg (FR); Roy M Golsteyn, Paris (FR); Daniel Louvard, Sceaux (FR)

Correspondence Address: YOUNG & THOMPSON, 745 SOUTH 23RD STREET 2ND FLOOR, ARLINGTON, VA, 22202

(21) Appl. No.: 10/239,431

(22) PCT Filed: Mar. 21, 2001
(86) PCT No.: PCT/FR01/00843

(30) Foreign Application Priority Data
Mar. 22, 2000 (FR) 00/03637

(51) Int. Cl. G01N 33/53; C07K 7/08; C07K 7/06

(52) U.S. Cl. 435/7.1; 530/328; 530/329; 530/330

(57) ABSTRACT

The invention concerns the use of proteins or peptides comprising one or several protein binding units of the Ena/VASP family, said proteins or peptides not binding with the Arp2/3 protein complex, in particular fragments of the ActA protein of Listeria monocytogenes, or proteins of the zyxin family, for preparing reagents for use in implementing a process detecting and screening molecules having an inhibiting or stimulating effect on the formation of actin cytoskeleton.

A subject of the present invention is peptide sequences comprising at least one unit binding to proteins of the Ena/VASP family, as well as the use of such sequences in particular within the scope of processes for detecting molecules having an effect of stimulation or inhibition on the formation of actin cytoskeleton.

The cells of our body are capable of moving around and sometimes they swell and divide into two sister cells. All of these movements are actin cytoskeleton based. At a multicellular stage, the cytoskeleton plays an essential role in the organisation of the body and in homeostasis. For example, cell migration is essential in embryogenesis and the immune response as well as during the repair of injuries where the cells migrate towards the damaged areas. These movements are dependant on the normal functioning of the actin cytoskeleton. The consequences of the disruption of the functioning of the cytoskeleton can be disastrous for the organism. In the metastatic process, for example, the absence of control by the cytoskeleton of tumour cells can cause their migration outwards from their normal location, allowing them to proliferate in other parts of the body, which makes the treatment of cancer extremely difficult.

The characterization of the proteins capable of polymerising actin, and the understanding of the mechanism by which this polymerisation generates a force, represent the key elements for understanding the functioning of the cytoskeleton in the cell. However, the dynamic properties of the cytoskeleton make its study extremely difficult. In addition, the approaches currently available to analyse the cytoskeleton are complicated or tedious.

The first stage of all the processes dependant on the cytoskeleton, such as movement, is the production of actin filaments, or F-actin. The mechanism for the formation of these biological polymers in the cell is still unknown, despite the identification of a number of actin binding proteins and the extensive study of actin polymerisation in vitro.

Wiskott-Aldrich Syndrome is a disease of the cytoskeleton. Human WASP protein, expressed from the WAS gene which is mutated in patients affected by this syndrome, just as N-WASP protein of bovine origin (which has approximately 45% sequence identity with human WASP protein), has thus been the subject of studies with the aim of clarifying the mechanism of the functioning of the cytoskeleton in the cell (Yarar et al., Current Biology, 9: 555-558 (1999); Rohatgi et al., Cell, 97: 221-231 (1999); Miki et al., The EMBO Journal, 15(19): 5326-5335 (1996)).

It has been shown that these WASP and N-WASP proteins interact with the Arp2/3 complex (protein complex involved in actin polymerisation), and thus induce actin polymerisation.

Therefore, it has been demonstrated that the WASP protein is sufficient to act on actin-based cellular motility, and that this function is under the control of the abovementioned Arp2/3 complex (Yarar et al. 1999). In order to carry out this demonstration, the authors of this article prepared microspheres coated with WASP protein and have shown that these microspheres polymerise actin, form actin tails, and are endowed with actin-based motility in cell extracts. In the cell extracts in which the Arp2/3 complex was suppressed, the microspheres coated with WASP protein cease motility and only have residual actin polymerisation activity.

Furthermore, a number of unicellular micro-organisms have their own independent means of movement but some pathogenic bacteria and viruses become mobile by using components of the cells that they infect.

The bacterium Listeria monocytogenes which infects man by food contamination, is one of these pathogens.

Listeria penetrates the cells, then recruits actin monomers at its surface, thus allowing them to form “comets” rich in actin F and to move (Sanger et al., Infection and Immunity, 60, 3609-3619 (1992); Tilney, L. G., DeRosier, D. J., Weber, A., and Tilney, M. S. (1992) Journal of Cell Biology, 118, 83-93).

Analysis of the human actin cytoskeleton has been greatly facilitated by the study of this Listeria (Beckerle, M. C., Cell 95, 741-748 (1998); Cossart P and Lecuit M., EMBO Journal 17, 3797-3806 (1998)).

ActA is a Listeria surface protein which is essential for its mobility (Domann et al., EMBO Journal 11, 1981-1990 (1992); Kocks, C., Gouin, E., Tabouret, M., Berche, P., Ohayon, H., and Cossart, P. (1992) Cell, 68, 521-531). It has been shown that polystyrene beads coated with ActA protein and placed in a cytoplasmic extract of Xenopus laevis eggs, were capable of moving around (Cameron et al., P.N.A.S. 96, 4908-4913 (1999)). This ActA protein is composed of an N-terminal region (delimited by the amino acids situated in positions 1 and 234 of FIG. 1) which interacts with the Arp 2/3 complex to induce actin nucleation activity (Welch M. D. et al, Science 281, 105-108 (1998)), followed by a large proline-rich domain (delimited by the amino acids situated in positions 235 and 584 of the peptide sequence represented in FIG. 1) of which it is supposed that it also plays a role within the scope of acceleration of the rate of actin assembly (Golsteyn R. M. et al., Journal of Cell Science, 110: 1893-1906 (1997)).

Human zyxin represents a protein the characterization of which has been facilitated by knowledge acquired in the course of studies carried out on Listeria (Beckerle, M. C., Bio Essays 19, 949-957 (1997)).

Zyxin represents the prototype of a new family of proteins which is located in actin-rich sites in superior eucaryotic cells (Petit, M. M., Mois, R., Schoenmakers E. F., Mandahl N., Van De Ven W. J. (1996) Genomic, 36, 118-129). By sequence analysis, other proteins of this family have been identified, such as the LPP protein (Lipoma Preferred Partner) the percentage of homology with zyxin of which is approximately 40% (Petit M. et al., Molecular Biology of the Cell, 11: 117-129), and the TRIP6 protein the percentage of homology with zyxin of which is approximately 35% (Yi, J., and Beckerle, M. C., Genomics 49, 314-316 (1998)).

These proteins of the zyxin family include a domain rich in proline residues of approximately 380 to 420 amino acids presenting a percentage of homology of approximately 20 to approximately 25% with the abovementioned proline-rich domain of the ActA protein.

The ActA protein and the proteins of the abovementioned zyxin family, bind using their proline-rich domain to the members of the Ena/VASP protein family, which comprises in particular the VASP protein (vasodilatator stimulated phosphoprotein), the Ena (in drosophila) and Mena (equivalent of the Ena protein in mammals) proteins, as well as the Evl protein (Chakraborty T. et al., EMBO Journal 14, 1314-1321 (1995); Reinhard M. et al, P.N.A.S. 92, 7956-7960 (1995)); Gertler F. B. et al, Cell 87: 227-239 (1996)).

The VASP protein will be involved in the organisation of the cytoskeleton as it binds to actin F and profilin, a 14 kDa protein which forms complexes with actin G (Reinhard M. et al., EMBO Journal 14, 1583-1589 (1995)), but this mechanism of action is not entirely clear.

The role of the Ena/VASP proteins, as well as that of zyxin and other proteins of the zyxin family in mammal cells is not clear at present.

The present invention results from the detection by the Inventors of the fact that there is, within the cells of the body a mechanism of actin polymerisation other than that involving the binding of proteins, such as those of the WASP family, to the Arp2/3 complex.

In fact, the Inventors have shown that proteins or protein fragments binding specifically to the proteins of the Ena/VASP family, but not binding with the Arp2/3 complex, are capable of polymerising actin, and allow the formation of the actin cytoskeleton in cell extracts when these proteins or protein fragments are adsorbed onto an appropriate solid support such as microspheres.

As opposed to the effects measured with beads coated with proteins binding to the Arp2/3 complex, in particular with beads coated with the abovementioned WASP family proteins, the beads coated with proteins or protein fragments binding specifically to the proteins of the Ena/VASP family according to the invention, and placed in lysed mammal, in particular human, cell supernatants, have made it possible for the inventors to show that:

actin polymerisation detected using the beads of the invention is inhibited by proteins or protein fragments binding specifically to the proteins of the Ena/VASP family (in particular by the ActA protein fragment hereafter designated ActA-Pro), whilst actin polymerisation detected using beads coated with WASP protein is not inhibited by the abovementioned proteins or protein fragments,

actin polymerisation detected using the beads of the invention is not inhibited by the WASP or N-WASP proteins, whilst the beads coated with WASP protein are inhibited by the WASP or N-WASP proteins,

the presence of the Arp2/3 complex in the abovementioned lysed-cell supernatants, do not appear essential for obtaining the effect of actin polymerisation on the beads of the invention, while it is obligatory in the case of beads coated with WASP protein,

the presence of proteins of the Ena/VASP family in the abovementioned lysed-cell supernatants, is necessary in order to obtain the effect of actin polymerisation on the beads of the invention, while it does not appear essential in the case of beads coated with WASP protein,

the beads of the invention are not likely to move around under the effect of the mechanism of actin polymerisation involving the proteins of the Ena/VASP family, whilst the beads coated with WASP protein are capable of moving around under the effect of the mechanism of actin polymerisation involving the Arp2/3 complex.

Furthermore, as a number of processes dependant on actin polymerisation require the recruitment and activation of the Arp2/3 complex, the Inventors have searched for the presence of this complex in the mitochondria carrying zyxin at their surface. No accumulation of the Arp2/3 proteins has been observed in the mitochondria, and in addition, the WASP protein does not inhibit polymerisation in the mitochondria in this test. These results allow the Inventors to conclude that the proteins of the zyxin family are sufficient to create polymerisation sites, this polymerisation requiring the presence of VASP.

An aim of the present invention is to provide new fragments, or polypeptide derivatives, of the ActA proteins and of the zyxin family, as well as the nucleotide sequences coding for these fragments.

An aim of the present invention is also to provide new processes of detecting or screening molecules having an effect on the formation of the cytoskeleton originating from the mechanism of interaction of the proteins of the Ena/VASP family with the ActA proteins and those of the zyxin family, in particular cytotoxic molecules or medicaments which can be used within the scope of the treatment of pathologies linked to an abnormal development of the cytoskeleton.

An aim of the invention is also to provide new reagents and kits for the implementation of the abovementioned processes.

A subject of the present invention is the use of proteins or peptides comprising one or more units binding to proteins of the Ena/VASP family, said proteins or peptides not binding with the Arp2/3 protein complex, and being capable of inducing actin polymerisation in vitro (namely of inducing the formation of F actin filaments in cell extracts or in comparable media, and in the absence of the Arp2/3 complex in these extracts or media, but in the presence of proteins of the Ena/VASP family), for the preparation of reagents which can be used within the scope of the implementation of a process for detecting or screening molecules having an effect of stimulation or inhibition on the formation of the actin cytoskeleton.

A subject of the invention is also the use of the abovementioned proteins or peptides, within the scope of the implementation of a process for detecting or screening molecules likely to be able to be used as medicaments in the treatment of pathologies linked to a dysfunction of the process of actin polymerisation within the scope of the formation of the actin cytoskeleton.

A more particular subject of the invention is the use of the abovementioned peptide fragments or derived sequences, within the scope of the implementation of a process for detecting or screening molecules having an inhibitory effect on the formation of the actin cytoskeleton, said molecules being likely to be used:

as medicaments in the treatment of metastatic cancers,

or as anti-parasitic antibiotics.

A more particular subject of the invention is use of the abovementioned peptide fragments or derived sequences, within the scope of the implementation of a detection process of the side-effects of molecules, in particular of medicaments or environmental molecules, namely a detection process of molecules likely to have a cytotoxic effect corresponding to an inhibition or stimulation of the formation of the actin cytoskeleton.

The abovementioned proteins or peptides used within the scope of the present invention, advantageously contain one or more units binding to proteins of the Ena/VASP family, said units comprising at least 5 to approximately 10 amino acids at least 3 of which are proline residues and, preferably, a phenylalanine residue. The abovementioned proteins or peptides also advantageously include at least two units binding to proteins of the Ena/VASP family.

A more particular subject of the invention is the abovementioned use of proteins or peptides defined above, comprising, as units binding to proteins of the Ena/VASP family, one or more of the following units of formula (I):

Phe-X₁-X₂-X₃-Pro-(X₄)ₙ (I)

in which:

n=0 or 1,

X₁ represents a proline or leucine residue,

X₂ represents a proline, leucine or serine residue,

X₃ represents a proline, isoleucine, or alanine residue,

X₄ represents a proline, leucine, or threonine residue,

on the condition that when n=0, at least two of X₁, X₂, X₃ represent a proline residue, and when n=1, at least two of X₁, X₂, X₃, and X₄ represent a proline residue.

The abovementioned proteins or peptides used within the scope of the present invention advantageously include two to four units of formula (I) defined above.

The abovementioned proteins or peptides used within the scope of the present invention also advantageously interact, via the units defined above, with the proteins of the Ena/VASP family, namely the abovementioned VASP protein, and/or the Ena protein, and/or the Mena protein, and/or the Evl protein, within the scope of actin polymerisation in the eucaryotic cells, in particular human, other mammal or insect cells.

The invention more particularly relates to the abovementioned use of proteins or peptides chosen from:

the fragments of the ActA protein of Listeria monocytogenes, said fragments of the ActA protein not binding with the Arp2/3 protein complex, and having the property of the ActA protein of binding to the proteins of the Ena/VASP family and polymerising the actin, or the derived sequences of these fragments, in particular by substitution, addition or suppression of one or more amino acids of these fragments, said derived sequences having the property of the ActA protein of binding to the proteins of the Ena/VASP family and polymerising the actin, and/or

the proteins of the zyxin family, or the fragments of the latter, or the derived sequences of these proteins or fragments, in particular by substitution, addition or suppression of one or more amino acids of these proteins or fragments, said fragments or derived sequences having the property of the proteins of the zyxin family of binding to the proteins of the Ena/VASP family and of polymerising the actin, and/or

vinculin from mammals, in particular human vinculin, or the fragments of the latter, or the sequences derived from this protein or fragments, in particular by substitution, addition or suppression of one or more amino acids of these proteins or fragments, said fragments or derived sequences having the property of proteins of the vinculin family of binding to the proteins of the Ena/VASP family and of polymerising the actin.

By binding to the VASP protein in the above and the following, is understood mainly binding of the electrostatic type, as well as Van der Waal's forces.

A more particular subject of the invention is the abovementioned use of fragments of the ActA protein of Listeria monocytogenes, designated SEQ ID NO 2 in the sequence listing below, the amino-terminal part of which binds with the Arp2/3 complex, namely the sequence corresponding to approximately the first 235 amino acids of SEQ ID NO 2, is suppressed or modified by substitution or suppression of one or more amino acids, in such a way that the fragments in question cannot bind with the Arp2/3 complex.

Thus, the invention more particularly relates to the abovementioned use:

of the sequence SEQ ID NO 4, corresponding to the fragment of 376 amino acids delimited by the amino acids situated in positions 235 and 610 of sequence SEQ ID NO 2,

of the sequence SEQ ID NO 6, corresponding to the fragment of 350 amino acids delimited by the amino acids situated in positions 235 and 584 of sequence SEQ ID NO 2.

A subject of the invention is also the abovementioned use of proteins of the zyxin family chosen from:

zyxin protein from mammals, in particular the murine zyxin represented by SEQ ID NO 8, the chicken zyxin represented by SEQ ID NO 10, and the human zyxin represented by SEQ ID NO 12,

LPP protein from mammals, in particular human LPP represented by SEQ ID NO 14,

TRIP6 protein from mammals, in particular human TRIP6 represented by SEQ ID NO 16, and murine TRIP6 represented by SEQ ID NO 18.

A more particular subject of the invention is the abovementioned use of fragments as defined above of proteins of the abovementioned zyxin family, and in particular of fragments chosen from:

Sequence SEQ ID NO 20, corresponding to the fragment of 374 amino acids delimited by the amino acids situated in positions 2 and 375 of sequence SEQ ID NO 8,

Sequence SEQ ID NO 22, corresponding to the fragment of 351 amino acids delimited by the amino acids situated in positions 1 and 351 of sequence SEQ ID NO 10,

Sequence SEQ ID NO 24, corresponding to the fragment of 380 amino acids delimited by the amino acids situated in positions 1 and 380 of sequence SEQ ID NO 12,

Sequence SEQ ID NO 26, corresponding to the fragment of 412 amino acids delimited by the amino acids situated in positions 3 and 414 of the sequence SEQ ID NO 14,

or the peptide sequences derived from the abovementioned peptide fragments, as defined above.

A more particular subject of the invention is also the abovementioned use of human zinculin designated SEQ ID NO 28 in the sequence listing below, or fragments as defined above of the latter, in particular sequence SEQ ID NO 30, corresponding to the fragment of 227 amino acids delimited by the amino acids situated in positions 840 and 1066 of the sequence SEQ ID NO 28.

A subject of the invention is also the abovementioned use of proteins, peptides, or sequences derived from the latter, as defined above, fused on the N-terminal or C-terminal side with one or more peptide sequences facilitating the detection and the purification of the abovementioned peptide fragments or derived sequences, without affecting the abovementioned property of the latter to polymerise actin. Among such peptide sequences fused to the peptide fragments, or to the sequences derived from the latter of the invention, glutathione-S-transferase can be mentioned (GST, described in Smith D. B. and Johnson K. S., Gene 67: 31-41 (1988)) fused to the N-terminal part of the abovementioned proteins or peptides or derived sequences, or those of epitopes recognised by specific antibodies, such as that of the myc9E10 epitope (described in Evan G. I. et al., Molecular and Cellular Biology 5: 3610-3616 (1985)) fused to the C-terminal part of the abovementioned proteins or peptides or derived sequences.

The invention also relates to the abovementioned peptide fragments as they are, namely more particularly sequences SEQ ID NO 4, SEQ ID NO 6, SEQ ID NO 20, SEQ ID NO 22, SEQ ID NO 24, SEQ ID NO 26, and SEQ ID NO 30, as well as the peptide sequences derived from the abovementioned peptide fragments, as defined above.

The invention also relates to the nucleotide sequences coding for the abovementioned peptide fragments, or for the peptide sequences derived from the latter, or also for the fusion proteins as described above.

A more particular subject of the invention is the following nucleotide sequences:

Sequence SEQ ID NO 3 coding for SEQ ID NO 4, sequence SEQ ID NO 5 coding for SEQ ID NO 6, sequence SEQ ID NO 19 coding for SEQ ID NO 20, sequence SEQ ID NO 21 coding for SEQ ID NO 22, sequence SEQ ID NO 23 coding for SEQ ID NO 24, sequence SEQ ID NO 25 coding for SEQ ID NO 26, sequence SEQ ID NO 29 coding for SEQ ID NO 30.

the nucleotide sequences derived by degeneration of the genetic code of the abovementioned nucleotide sequences, and coding for the abovementioned proteins or peptides,

the nucleotide sequences derived from the abovementioned nucleotide sequences, and coding for the sequences derived from said proteins or peptides as defined above.

A subject of the invention is also the vectors, in particular the plasmids, containing a nucleotide sequence as defined above.

The invention also relates to the host cells transformed by an abovementioned vector, said cells expressing the abovementioned peptide fragments, or the derived sequences described above, in recombinant form. The abovementioned host cells are advantageously chosen from the following: Escherichia coli DH5α and Escherichia coli BL21.

A subject of the invention is also reagents for the implementation of a process of detecting or screening molecules having an effect of inhibition or stimulation on the formation of the actin cytoskeleton, said reagent comprising at least one protein or peptide as defined above, bound or adsorbed to a support likely to permit actin polymerisation, when said support bound to said peptide is placed in a medium containing the elements necessary for actin polymerisation, in particular when said support is added to an extract prepared from lysed mammal cell supernatants, or in a medium containing mainly the proteins of the Ena/VASP family, cofilin, and capping proteins, but not necessarily containing the Arp2/3 complex.

A more particular subject of the invention is reagents as defined above, chosen from microspheres the diameter of which is between approximately 100 and approximately 10 000 nm, the material constituting the microspheres being itself chosen from polystyrene or latex, said microspheres each containing approximately 5 000 to approximately 50 000 molecules of abovementioned protein or peptide or a derived sequence according to the invention.

The abovementioned protein or peptide, or their derived sequence, are advantageously adsorbed or bound in a covalent manner with a reactive site at the surface of said microspheres, said reagent being obtained by simple mixture of said microspheres with the protein or the peptide or with their derived sequence.

A subject of the invention is also a process of detecting or screening molecules having an effect of inhibition or stimulation on the formation of the actin cytoskeleton, said process comprising:

a stage of placing the tested molecule in the presence of a reagent as defined above, in a medium containing actin and the elements necessary for actin polymerisation defined above, in particular in an extract of lysed cell supernatant,

followed by the optional detection of an inhibition or an activation of the process of actin polymerisation at the surface of said reagent, in comparison to a control (namely a medium as described above not containing the tested molecule, and in which is found said reagent), corresponding respectively to an effect of inhibition or stimulation of the tested molecule on the formation of the actin cytoskeleton by the mechanism involving the abovementioned protein or peptide bond or their derived sequence, with a protein of the Ena protein/VASP family.

The abovementioned medium in which the tested molecule is placed in the presence of said reagent, advantageously contains a compound labelled in particular by fluorescence, making it possible to detect actin polymerisation on said reagent. By way of illustration, the abovementioned labelled compound is a fluorescent derivative of actin, such as actin-rhodamine (commercially available), making it possible to visualize actin polymerisation by epifluorescence microscopy.

The invention also relates to a process as defined above, of detecting or screening molecules having an effect of inhibition or stimulation on the formation of the actin cytoskeleton, said process in addition comprising stages of the process defined above:

a stage of placing, in a medium containing actin and the elements necessary for actin polymerisation i.e. the Arp2/3 complex, in particular in an extract of lysed cell supernatant, the tested molecule with a reagent comprising WASP family proteins in the eucaryotic cells, in particular human or of other mammal cells, or insect cells, or from micro-organisms such as yeasts, or peptide fragments of these WASP family proteins, said peptide fragments having the property of the WASP family proteins to polymerise the actin by inducing cellular motility, or peptide sequences derived from WASP family proteins or the abovementioned peptide fragments, in particular by substitution of one or more amino acids of these fragments, said derived sequences having the abovementioned property of the WASP family proteins and said fragments of the latter, said abovementioned WASP family proteins, or peptide fragments or derived sequences, being bound or adsorbed to a support as defined above,

followed by the optional detection of an inhibition or an activation of the process of actin polymerisation at the surface of said reagent, in comparison to a control, corresponding respectively to an effect of inhibition or of stimulation of the tested molecule on the formation of the actin cytoskeleton by the mechanism involving the bonding of said WASP family proteins, or abovementioned peptide fragments or derived sequences, with the Arp2/3 complex.

By WASP family proteins, is understood, in the above and in the following, the protein produced by the WAS gene mutated within the scope of Wiskott-Aldrich syndrome in man, as well as the proteins of human or non-human origin, presenting at least approximately 45% homology with the abovementioned human WASP protein, and being involved in the process of cellular actin polymerisation, and, if appropriate, of cellular motility.

The abovementioned WASP family proteins also possess the common characteristic of possessing at least three main domains:

a WH1/Scar domain in the N-terminal part; this domain has structural characteristics similar to a pleckstrin homology domain (or pH domain), and it is assumed interacts with the polymerised actin and the phospholipids,

a proline-rich domain,

a WH2/A domain which is divided into three sub-domains, namely the verprolin homology subdomain, the cofilin homology subdomain, and an acid sub-domain.

The abovementioned WASP family proteins and the peptide fragments of the latter used within the scope of the abovementioned process of the present invention, are advantageously chosen from the WASP, N-WASP, Scar and Las17 proteins or their fragments, or the peptide sequences derived from the abovementioned peptide fragments as defined above.

A more particular subject of the invention is the abovementioned process in which the peptide fragments of WASP family proteins are chosen from the fragments:

of human or other mammalian WASP protein, in particular bovine or murine WASP protein,

of the human or other mammalian N-WASP protein, in particular bovine or rat N-WASP protein,

proteins of the Scar sub-family, such as the mouse or human Scar1/WAVE protein of Dictyostellium discoideum, or Caenorhabditis elegans, or Drosophila melanogaster,

proteins of the Las17 sub-family of micro-organisms, in particular of yeasts, such as the Las17/Bee1 protein of Saccharomyces cerevisiae, or the homologous WASP protein (Wsp1p) of Schizosaccharomyces pombe.

The abovementioned peptide fragments are advantageously chosen from those comprising:

the verprolin homology domain contained in the WASP family proteins, or in a protein derived from the latter, or at least one of the two verprolin-homologous sequences when said WASP family proteins contain two of these sequences, or a peptide sequence derived from the abovementioned domain, in particular by substitution, addition or suppression of one or more amino acids, and retaining the property of this domain of binding to actin,

and the cofilin homology domain contained in the WASP family proteins or in a protein derived from the latter, or a peptide sequence derived from the abovementioned domain, in particular by substitution, addition or suppression of one or more amino acids, and retaining the property of this domain to occur within the scope of actin polymerisation.

If appropriate, the abovementioned peptide fragments used within the scope of the present invention, also contain the C-terminal acid segment of said WASP proteins or derivatives.

The abovementioned peptide fragments advantageously do not contain the pleckstrin homology domain, and/or the Cdc42 binding domain, and/or the proline-rich domain, defined above of said WASP family proteins.

A more particular subject of the invention is the abovementioned use in the process defined above, of peptide fragments of WASP family proteins of human origin.

The peptide fragments of the WASP family proteins of human origin are advantageously chosen from the fragments of human WASP protein comprising:

the verprolin homology domain delimited by the amino acids situated in positions 430 and 446 of the peptide sequence of human WASP protein represented by SEQ ID NO 31, or a peptide sequence derived from the abovementioned domain as defined above,

and the cofilin homology domain delimited by the amino acids situated in positions 469 and 487 of the peptide sequence of human WASP protein represented by SEQ ID NO 31, or a peptide sequence derived from the abovementioned domain as defined above.

Preferably, the fragments of the abovementioned human WASP protein are chosen from the following:

the fragments of which the N-terminal amino acid corresponds to that situated in one of positions 404 to 430 of SEQ ID NO 31, and the C-terminal amino acid corresponds to that situated in one of positions 487 to 502 of SEQ ID NO 31,

the fragment of 99 amino acids delimited by the amino acids situated in positions 404 and 502 of SEQ ID NO 31,

the fragment of 84 amino acids delimited by the amino acids situated in positions 404 and 487 of SEQ ID NO 31,

the fragment of 73 amino acids delimited by the amino acids situated in positions 430 and 502 of SEQ ID NO 31,

the fragment of 58 amino acids delimited by the amino acids situated in positions 430 and 487 of SEQ ID NO 31,

or the peptide sequences derived from the abovementioned peptide fragments, in particular by substitution, addition or suppression of one or more amino acids of these fragments, said derived sequences having the property defined above of the WASP family proteins and of said fragments of the latter.

The peptide fragments of the WASP family proteins of human origin used in the process defined above, are advantageously chosen from the fragments of human N-WASP protein comprising:

the verprolin homologous sequence delimited by the amino acids situated in positions 405 and 421 of the peptide sequence of the human N-WASP protein represented by SEQ ID NO 32, or a peptide sequence derived from the abovementioned domain as defined above,

and/or the verprolin homologous sequence delimited by the amino acids situated in positions 433 and 449 of the peptide sequence of the human N-WASP protein represented by SEQ ID NO 32, or a peptide sequence derived from the abovementioned domain as defined above,

and the cofilin homology domain contained in the abovementioned N-WASP protein, namely the domain delimited by the amino acids situated in positions 470 and 488 of the peptide sequence of human N-WASP protein represented by SEQ ID NO 32, or a peptide sequence derived from the abovementioned domain as defined above.

Preferably, the fragments of the abovementioned human N-WASP protein are chosen from the following:

the fragments of which the N-terminal amino acid corresponds to that situated in one of positions 392 to 433 of SEQ ID NO 32, and the C-terminal amino acid corresponds to that situated in one of positions 488 to 505 of SEQ ID NO 32,

the fragment of 114 amino acids delimited by the amino acids situated in positions 392 and 505 of SEQ ID NO 32,

the fragment of 97 amino acids delimited by the amino acids situated in positions 392 and 488 of SEQ ID NO 32,

the fragment of 101 amino acids delimited by the amino acids situated in positions 405 and 505 of SEQ ID NO 32,

the fragment of 84 amino acids delimited by the amino acids situated in positions 405 and 488 of SEQ ID NO 32,

the fragment of 73 amino acids delimited by the amino acids situated in positions 433 and 505 of SEQ ID NO 32,

the fragment of 56 amino acids delimited by the amino acids situated in positions 433 and 488 of SEQ ID NO 32,

or the peptide sequences derived from the abovementioned peptide fragments, in particular by substitution, addition or suppression of one or more amino acids of these fragments, said derived sequences having the property defined above of the WASP family proteins and of said fragments of the latter.

The peptide fragments of the WASP family proteins of human origin used in the process defined above, are advantageously chosen from the fragments of human Scar1 protein comprising:

the verprolin homology domain delimited by the amino acids situated in positions 497 and 513 of the peptide sequence of the human Scar1 protein represented by SEQ ID NO 33, or a peptide sequence derived from the abovementioned domain as defined above,

and the cofilin homology domain delimited by the amino acids situated in positions 531 and 546 of the peptide sequence of human Scar1 protein represented by SEQ ID NO 33, or a peptide sequence derived from the abovementioned domain as defined above.

Preferably, the abovementioned fragments of human Scar1 protein are chosen from the following:

the fragments of which the N-terminal amino acid corresponds to that situated in one of positions 443 to 497 of SEQ ID NO 33, and the C-terminal amino acid corresponds to that situated in one of positions 546 to 559 of SEQ ID NO 33,

the fragment of 117 amino acids delimited by the amino acids situated in positions 443 and 559 of SEQ ID NO 33,

the fragment of 104 amino acids delimited by the amino acids situated in positions 443 and 546 of SEQ ID NO 33,

the fragment of 63 amino acids delimited by the amino acids situated in positions 497 and 559 of SEQ ID NO 33,

the fragment of 50 amino acids delimited by the amino acids situated in positions 497 and 546 of SEQ ID NO 33,

or the peptide sequences derived from the abovementioned peptide fragments, in particular by substitution, addition or suppression of one or more amino acids of these fragments, said derived sequences having the property defined above of the WASP family proteins and said fragments of the latter.

A more particular subject of the invention is the abovementioned use of peptide fragments of the WASP family proteins of non-human origin.

The peptide fragments of WASP family proteins of non-human origin used in the process defined above, are advantageously chosen from the fragments of WASP family proteins of non-human mammals, such as:

the fragments of the murine WASP protein, themselves chosen from:

those comprising:

the verprolin homology domain delimited by the amino acids situated in positions 448 and 465 of the peptide sequence of the murine WASP protein represented by SEQ ID NO 34, or a peptide sequence derived from the abovementioned domain as defined above,

and the cofilin homology domain delimited by the amino acids situated in positions 487 and 505 of the peptide sequence of the murine WASP protein represented by SEQ ID NO 34, or a peptide sequence derived from the abovementioned domain as defined above,

the fragments of which the N-terminal amino acid corresponds to that situated in one of positions 420 to 448 of SEQ ID NO 34, and the C-terminal amino acid corresponds to that situated in one of positions 505 to 520 of SEQ ID NO 34,

the fragment of 101 amino acids delimited by the amino acids situated in positions 420 and 520 of SEQ ID NO 34,

the fragment of 86 amino acids delimited by the amino acids situated in positions 420 and 505 of SEQ ID NO 34,

the fragment of 73 amino acids delimited by the amino acids situated in positions 448 and 520 of SEQ ID NO 34,

the fragment of 58 amino acids delimited by the amino acids situated in positions 448 and 505 of SEQ ID NO 34,

the fragments of rat N-WASP protein, themselves chosen from:

those comprising:

the verprolin homologous sequence delimited by the amino acids situated in positions 401 and 417 of the peptide sequence of the rat N-WASP protein represented by SEQ ID NO 35, or a peptide sequence derived from the abovementioned domain as defined above,

and/or the verprolin homologous sequence delimited by the amino acids situated in positions 429 and 444 of the peptide sequence of the rat N-WASP protein represented by SEQ ID NO 35, or a peptide sequence derived from the abovementioned domain as defined above,

and the cofilin homology domain contained in the abovementioned N-WASP protein, namely the domain delimited by the amino acids situated in positions 466 and 484 of the peptide sequence of the rat N-WASP protein represented by SEQ ID NO 35, or a peptide sequence derived from the abovementioned domain as defined above,

the fragments of which the N-terminal amino acid corresponds to that situated in one of positions 401 to 429 of SEQ ID NO 35, and the C-terminal amino acid corresponds to that situated in one of positions 484 to 501 of SEQ ID NO 35,

the fragment of 101 amino acids delimited by the amino acids situated in positions 401 and 501 of SEQ ID NO 35,

the fragment of 84 amino acids delimited by the amino acids situated in positions 401 and 484 of SEQ ID NO 35,

the fragment of 73 amino acids delimited by the amino acids situated in positions 429 and 501 of SEQ ID NO 35,

the fragment of 56 amino acids delimited by the amino acids situated in positions 429 and 484 of SEQ ID NO 35,

the fragments of the bovine N-WASP protein, themselves chosen from:

those comprising:

the verprolin homology domain delimited by the amino acids situated in positions 405 and 421 of the peptide sequence of the bovine N-WASP protein represented by SEQ ID NO 36, or a peptide sequence derived from the abovementioned domain as defined above,

and/or the verprolin homology domain delimited by the amino acids situated in positions 433 and 488 of the peptide sequence of the bovine N-WASP protein represented by SEQ ID NO 36, or a peptide sequence derived from the abovementioned domain as defined above,

and the cofilin homology domain delimited by the amino acids situated in positions 470 and 488 of the peptide sequence of the bovine N-WASP protein represented by SEQ ID NO 36, or a peptide sequence derived from the abovementioned domain as defined above,

the fragments of which the N-terminal amino acid corresponds to that situated in one of positions 405 to 433 of SEQ ID NO 36, and the C-terminal amino acid corresponds to that situated in one of positions 488 to 505 of SEQ ID NO 36,

the fragment of 101 amino acids delimited by the amino acids situated in positions 405 and 505 of SEQ ID NO 36,

the fragment of 84 amino acids delimited by the amino acids situated in positions 405 and 488 of SEQ ID NO 36,

the fragment of 73 amino acids delimited by the amino acids situated in positions 433 and 488 of SEQ ID NO 36,

the fragment of 56 amino acids delimited by the amino acids situated in positions 433 and 488 of SEQ ID NO 36,

The peptide fragments of the WASP family proteins of non-human origin used in the process defined above, are advantageously chosen from the fragments of the WASP family proteins of micro-organisms, such as:

the fragments of the Las17 protein of Saccharomyces cerevisiae, themselves chosen from:

those comprising:

the verprolin homology domain delimited by the amino acids situated in positions 447 and 466 of the peptide sequence of the Las17 protein represented by SEQ ID NO 37, or a peptide sequence derived from the abovementioned domain as defined above,

and the cofilin homology domain delimited by the amino acids situated in positions 607 and 624 of the peptide sequence of the Las17 protein represented by SEQ ID NO 37, or a peptide sequence derived from the abovementioned domain as defined above,

the fragments of which the N-terminal amino acid corresponds to that situated in one of positions 422 to 447 of SEQ ID NO 37, and the C-terminal amino acid corresponds to that situated in one of positions 624 to 633 of SEQ ID NO 37,

the fragment of 212 amino acids delimited by the amino acids situated in positions 422 and 633 of SEQ ID NO 37,

the fragment of 203 amino acids delimited by the amino acids situated in positions 422 and 624 of SEQ ID NO 37,

the fragment of 187 amino acids delimited by the amino acids situated in positions 447 and 633 of SEQ ID NO 37,

the fragment of 178 amino acids delimited by the amino acids situated in positions 447 and 624 of SEQ ID NO 37,

the fragments of the WASP homologous protein (Wsp1p) of Schizosaccharomyces pombe, themselves chosen from:

those comprising:

the verprolin homology domain delimited by the amino acids situated in positions 501 and 517 of the peptide sequence of the WASP homologous protein (Wsp1p) of Schizosaccharomyces pombe represented by SEQ ID NO 38, or a peptide sequence derived from the abovementioned domain as defined above,

and the cofilin homology domain delimited by the amino acids situated in positions 548 and 565 of the peptide sequence of the WASP homologous protein (Wsp1p) of Schizosaccharomyces pombe represented by SEQ ID NO 38, or a peptide sequence derived from the abovementioned domain as defined above,

the fragments of which the N-terminal amino acid corresponds to that situated in one of positions 477 to 501 of SEQ ID NO 38, and the C-terminal amino acid corresponds to that situated in one of positions 565 to 574 of SEQ ID NO 38,

the fragment of 98 amino acids delimited by the amino acids situated in positions 477 and 574 of SEQ ID NO 38,

the fragment of 89 amino acids delimited by the amino acids situated in positions 477 and 565 of SEQ ID NO 38,

the fragment of 74 amino acids delimited by the amino acids situated in positions 501 and 574 of SEQ ID NO 38,

the fragment of 65 amino acids delimited by the amino acids situated in positions 501 and 565 of SEQ ID NO 38.

The peptide fragments of the WASP family proteins of human or non-human origin used in the process defined above, are advantageously chosen from the peptide sequences derived from the abovementioned peptide fragments, in particular by substitution, addition or suppression of one or more amino acids of these fragments, said derived sequences having the property defined above of the WASP family proteins and said fragments of the latter.

A subject of the invention is also the application of the process as defined above, in the detection or the screening of molecules:

likely to be able to be used as medicaments in the treatment of pathologies linked to a dysfunction of the process of actin polymerisation within the scope of the formation of the actin cytoskeleton, in particular as medicaments in the treatment of metastatic cancers, or as anti-parasitic antibiotics,

or likely to have a cytotoxic effect corresponding to an inhibition or a stimulation of the formation of the actin cytoskeleton.

A subject of the invention is also a kit for the implementation of an abovementioned process, comprising

a reagent as defined above,

if appropriate a reagent comprising WASP family proteins in the eucaryotic cells, or peptide sequences derived from the WASP family proteins or peptide fragments defined above of these proteins or derived sequences, bound or adsorbed to a support as defined above,

if appropriate a labelled compound making it possible to visualize actin polymerisation, in particular actin labelled by fluorescence,

if appropriate a suitable medium containing the elements necessary for actin polymerisation, in particular an extract of lysed cells.

The invention is further illustrated using the detailed description which follows of the preparation of microspheres coated with a peptide fragment of the ActA protein, and the detection of actin polymerisation at the surface of these microspheres in an extract of cell supernatant.

I) Preparation of the GST-ActA-Pro Beads

The sequence coding for the amino-terminal domain (1-234) of the cDNA coding for the ActA protein of the Listeria monocytogenes bacterium was suppressed, as well as the part coding for the last 20 amino acids of the carboxy-terminal domain (transmembrane anchoring of the protein). The sequence of remaining DNA, coding for the central part (proline rich) and the carboxy-terminal part of ActA, was introduced into the pGEX2T vector (Pharmacia), downstream of the sequence coding for glutathione-S-transferase (GST), generating the pGEX2T-ActA-Pro plasmid. The GST domain was chosen as it facilitates the purification of the protein. This recombinant protein is composed of GST domains (237 residues) and proline rich and carboxy-terminal central parts of ActA (350 residues corresponding to SEQ ID NO 6).

II) Purification and Characterization of the GST-ActA-Pro Protein.

E. coli bacteria (strain BL21) were transformed with the pGEX2T-ActA-Pro plasmid. The bacteria were cultivated in LB standard medium containing the antibiotic ampicillin for maintaining under selection pressure the bacteria comprising the plasmid. The bacteria were cultivated in suspension at 37° C. until the culture reached an optical density of 0.6 at 600 nm. Then, isopropylthio-B-D-galactoside (IPTG) was added to the medium at a final concentration of 1 mM to induce the production of the protein. After 1 hour, the bacteria were collected by centrifugation and the pellets stored at −80° C. The pellets were defrosted and added to extraction buffer (saline solution buffered with phosphate pH 8, 300 mM NaCl, 2 mM EDTA (ethylenediamine-tetra-acetic acid), 1 mM DTT, 0.5% Triton X-100, containing 1 μg/ml of each of the following protease inhibitors, leupeptin, benzamidine, pepstatin, at a ratio of 1 gr of pellet per 10 volumes of extraction buffer. The suspension was sonicated until it was no longer viscous. The extract was centrifuged at 20 000×g for 10 minutes at 4° C. and the supernatant containing the GST-ActA-Pro protein was retained. The ActA-pro protein was purified from the bacterial extract by affinity chromatography on resin coupled to Glutathione (Pharmacia) and eluted with 10 mM reduced glutathione according to the manufacturers' recommendations. Purification was confirmed by analysis of the GST-Acta-Pro by electrophoresis on acrylamide gel.

The GST-ActA-Pro protein was adsorbed onto 500 nm latex beads (Polyscience Inc, 400 Valley Road, Warrington Pa., USA) according to the manufacturers' instructions. These beads, added to the extracts prepared from cells, are capable of nucleating the actin.

1. Use of proteins or peptides comprising one or more units binding to proteins of the Ena/VASP family, said proteins or peptides not binding with the Arp2/3 protein complex, and being capable of inducing actin polymerisation in vitro, for the preparation of reagents which can be used within the scope of the implementation of a process of detecting or screening molecules having an effect of inhibition or stimulation on the formation of the actin cytoskeleton.

2. Use of proteins or peptides according to claim 1, containing one or more units binding to proteins of the Ena/VASP family, said units comprising at least 5 to approximately 10 amino acids i.e. at least 3 proline residues.

3. Use of proteins or peptides according to claim 1 or 2, containing one or more units binding to proteins of the Ena/VASP family, said units comprising at least 5 to approximately 10 amino acids i.e. at least 3 proline residues and a phenylalanine residue.

4. Use of proteins or peptides according to one of claims 1 to 3, comprising at least two units binding to proteins of the Ena/VASP family.

5. Use of proteins or peptides according to one of claims 1 to 4, comprising one or more following units of formula (I):
Phe-X₁-X₂-X₃-Pro-(X₄)ₙ (I)
in which:
n=0 or 1,
X₁ represents a proline or leucine residue,
X₂ represents a proline, leucine or serine residue,
X₃ represents a proline, isoleucine, or alanine residue,
X₄ represents a proline, leucine, or threonine residue,
on condition that when n=0, at least two of X₁, X₂, X₃ represent a proline residue, and when n=1, at least two of X₁, X₂, X₃, and X₄ represent a proline residue.

6. Use according to one of claims 1 to 5 of peptides chosen from:
the fragments of the ActA protein of Listeria monocytogenes, said fragments of the ActA protein not binding with the Arp2/3 protein complex, and having the property of the ActA protein of binding to the proteins of the Ena/VASP family and of polymerising the actin, or the sequences derived from these fragments, in particular by substitution, addition or suppression of one or more amino acids of these fragments, said derived sequences having the property of the ActA protein of binding to the proteins of the Ena/VASP family and polymerising the actin, and/or
the proteins of the zyxin family, or the fragments of the latter, or the sequences derived from these proteins or fragments, in particular by substitution, addition or suppression of one or more amino acids of these proteins or fragments, said derived fragments or sequences having the property of the proteins of the zyxin family of binding to the proteins of the Ena/VASP family and of polymerising the actin,
mammal vinculin, in particular human vinculin, or the fragments of the latter, or the sequences derived from this protein or fragments, in particular by substitution, addition or suppression of one or more amino acids of these proteins or fragments, said derived fragments or sequences having the property of the proteins of the vinculin family of binding to the proteins of the Ena/VASP family and of polymerising the actin.

7. Use according to one of claims 1 to 6, of peptides chosen from the following peptide fragments:
the sequence SEQ ID NO 4, corresponding to the fragment of 376 amino acids delimited by the amino acids situated in positions 235 and 610 of sequence SEQ ID NO 2,
the sequence SEQ ID NO 6, corresponding to the fragment of 350 amino acids delimited by the amino acids situated in positions 235 and 584 of the sequence SEQ ID NO 2,
the sequence SEQ ID NO 20, corresponding to the fragment of 374 amino acids delimited by the amino acids situated in positions 2 and 375 of the sequence SEQ ID NO8,
the sequence SEQ ID NO 22, corresponding to the fragment of 351 amino acids delimited by the amino acids situated in positions 1 and 351 of the sequence SEQ ID NO 10,
the sequence SEQ ID NO 24, corresponding to the fragment of 380 amino acids delimited by the amino acids situated in positions 1 and 380 of sequence SEQ ID NO 12,
the sequence SEQ ID NO 26, corresponding to the fragment of 412 amino acids delimited by the amino acids situated in positions 3 and 414 of the sequence SEQ ID NO 14,
the sequence SEQ ID NO 30, corresponding to the fragment of 227 amino acids delimited by the amino acids situated in positions 840 and 1066 of the sequence SEQ ID NO 28,
or the peptide sequences derived from the abovementioned peptide fragments, as defined in claim 6.

8. Reagent for the implementation of a process of detecting or screening molecules having an effect of stimulation or inhibition on the formation of the actin cytoskeleton, said reagent comprising at least one protein or peptide as defined in one of claims 1 to 7, bound or adsorbed to a support likely to allow actin polymerisation, when said support bound to said protein or said peptide is placed in a medium containing the elements necessary for actin polymerisation, in particular when said support is added to an extract prepared from lysed mammal cell supernatants.

9. Reagent according to claim 8, characterized in that it is chosen from microspheres the diameter of which is between approximately 100 and approximately 10 000 nm, the material constituting the microspheres being itself chosen from polystyrene or latex, said microspheres each containing approximately 5 000 to approximately 50 000 molecules of peptide or derived sequence defined in one of claims 1 to 7.

10. Process for the detection or screening of molecules having an effect of inhibition or stimulation on the formation of the actin cytoskeleton, said process comprising:
a stage of placing the tested molecule in the presence of a reagent according to claim 8 or 9, in a medium containing actin and the elements necessary for actin polymerisation, in particular in an extract of lysed cell supernatant,
followed by the optional detection of inhibition or activation of the process of actin polymerisation at the surface of said reagent, in comparison to a control, corresponding respectively to an effect of inhibition or of stimulation of the tested molecule on the formation of the actin cytoskeleton by the mechanism involving the binding of the abovementioned protein or peptide or their derived sequence with a protein of the Ena protein/VASP family.

11. Process according to claim 10, of detecting or screening molecules having an effect of inhibition or stimulation on the formation of the actin cytoskeleton, said process comprising in addition stages of the process defined in claim 10:
a stage of placing, in a medium containing actin and the elements necessary for actin polymerisation i.e. the Arp2/3 complex, in particular in an extract of lysed-cell supernatant, the tested molecule in the presence of a reagent comprising WASP family proteins in the eucaryotic cells, in particular human, other mammal or insect cells, or micro-organisms such as yeasts, or peptide fragments of these WASP family proteins, said peptide fragments having the property of the WASP family proteins of polymerising the actin by inducing cellular motility, or peptide sequences derived from the WASP family proteins or the abovementioned peptide fragments, in particular by substitution of one or more amino acids of these fragments, said derived sequences having the abovementioned property of the WASP family proteins and of said fragments of the latter, said WASP family proteins, or abovementioned peptide fragments or derived sequences, being bound or adsorbed to a support as defined above,
followed by the optional detection of an inhibition or activation of the process of actin polymerisation at the surface of said reagent, in comparison to a control, corresponding respectively to an effect of inhibition or of stimulation of the tested molecule on the formation of the actin cytoskeleton by the mechanism involving the binding of said WASP family proteins, or abovementioned peptide fragments or derived sequences, with the Arp2/3 complex.

12. Process according to claim 11, characterized in that the WASP family proteins used are chosen from:
human or other mammal WASP protein, such as bovine or murine WASP protein,
human or other mammal N-WASP protein, such as bovine or rat N-WASP protein,
the proteins of the Scar sub-family, such as the mouse or human Scar1/WAVE protein of Dictyostellium discoideum, or Caenorhabditis elegans, or Drosophila melanogaster,
the proteins of the Las17 sub-family of micro-organisms, in particular yeasts, such as the Las17/Bee1 protein of Saccharomyces cerevisiae, or the homologous WASP protein (Wsp1p) of Schizosaccharomyces pombe.
or the peptide sequences derived from the abovementioned proteins as defined in claim 11.

13. Process according to claim 11, characterized in that the fragments of the WASP family proteins used are chosen from:
the following fragments of human WASP protein:
the fragments of which the N-terminal amino acid corresponds to that situated in one of positions 404 to 430 of SEQ ID NO 31, and the C-terminal amino acid corresponds to that situated in one of positions 487 to 502 of SEQ ID NO 31,
the fragment of 99 amino acids delimited by the amino acids situated in positions 404 and 502 of SEQ ID NO 31,
the fragment of 84 amino acids delimited by the amino acids situated in positions 404 and 487 of SEQ ID NO 31,
the fragment of 73 amino acids delimited by the amino acids situated in positions 430 and 502 of SEQ ID NO 31,
the fragment of 58 amino acids delimited by the amino acids situated in positions 430 and 487 of SEQ ID NO 31,
the following fragments of human N-WASP protein:
the fragments of which the N-terminal amino acid corresponds to that situated in one of positions 392 to 433 of SEQ ID NO 32, and the C-terminal amino acid corresponds to that situated in one of positions 488 to 505 of SEQ ID NO 32,
the fragment of 114 amino acids delimited by the amino acids situated in positions 392 and 505 of SEQ ID NO 32,
the fragment of 97 amino acids delimited by the amino acids situated in positions 392 and 488 of SEQ ID NO 32,
the fragment of 101 amino acids delimited by the amino acids situated in positions 405 and 505 of SEQ ID NO 32,
the fragment of 84 amino acids delimited by the amino acids situated in positions 405 and 488 of SEQ ID NO 32,
the fragment of 73 amino acids delimited by the amino acids situated in positions 433 and 505 of SEQ ID NO 32,
the fragment of 56 amino acids delimited by the amino acids situated in positions 433 and 488 of SEQ ID NO 32,
the following fragments of human Scar1 protein:
the fragments of which the N-terminal amino acid corresponds to that situated in one of positions 443 to 497 of SEQ ID NO 33, and the C-terminal amino acid corresponds to that situated in one of positions 546 to 559 of SEQ ID NO 33,
the fragment of 117 amino acids delimited by the amino acids situated in positions 443 and 559 of SEQ ID NO 33,
the fragment of 104 amino acids delimited by the amino acids situated in positions 443 and 546 of SEQ ID NO 33,
the fragment of 63 amino acids delimited by the amino acids situated in positions 497 and 559 of SEQ ID NO 33,
the fragment of 50 amino acids delimited by the amino acids situated in positions 497 and 546 of SEQ ID NO 33,
the following fragments of murine WASP protein:
the fragments of which the N-terminal amino acid corresponds to that situated in one of positions 420 to 448 of SEQ ID NO 34, and the C-terminal amino acid corresponds to that situated in one of positions 505 to 520 of SEQ ID NO 34,
the fragment of 101 amino acids delimited by the amino acids situated in positions 420 and 520 of SEQ ID NO 34,
the fragment of 86 amino acids delimited by the amino acids situated in positions 420 and 505 of SEQ ID NO 34,
the fragment of 73 amino acids delimited by the amino acids situated in positions 448 and 520 of SEQ ID NO 34,
the fragment of 58 amino acids delimited by the amino acids situated in positions 448 and 505 of SEQ ID NO 34,
the following fragments of rat N-WASP protein:
the fragments of which the N-terminal amino acid corresponds to that situated in one of positions 401 to 429 of SEQ ID NO 35, and the C-terminal amino acid corresponds to that situated in one of positions 484 to 501 of SEQ ID NO 35,
the fragment of 101 amino acids delimited by the amino acids situated in positions 401 and 501 of SEQ ID NO 35,
the fragment of 84 amino acids delimited by the amino acids situated in positions 401 and 484 of SEQ ID NO 35,
the fragment of 73 amino acids delimited by the amino acids situated in positions 429 and 501 of SEQ ID NO 35,
the fragment of 56 amino acids delimited by the amino acids situated in positions 429 and 484 of SEQ ID NO 35,
the following fragments of bovine N-WASP protein:
the fragments of which the N-terminal amino acid corresponds to that situated in one of positions 405 to 433 of SEQ ID NO 36, and the C-terminal amino acid corresponds to that situated in one of positions 488 to 505 of SEQ ID NO 36,
the fragment of 101 amino acids delimited by the amino acids situated in positions 405 and 505 of SEQ ID NO 36,
the fragment of 84 amino acids delimited by the amino acids situated in positions 405 and 488 of SEQ ID NO 36,
the fragment of 73 amino acids delimited by the amino acids situated in positions 433 and 488 of SEQ ID NO 36,
the fragment of 56 amino acids delimited by the amino acids situated in positions 433 and 488 of SEQ ID NO 36,
the following fragments of the Las17 protein of Saccharomyces cerevisiae:
the fragments of which the N-terminal amino acid corresponds to that situated in one of positions 422 to 447 of SEQ ID NO 37, and the C-terminal amino acid corresponds to that situated in one of positions 624 to 633 of SEQ ID NO 37,
the fragment of 212 amino acids delimited by the amino acids situated in positions 422 and 633 of SEQ ID NO 37,
the fragment of 203 amino acids delimited by the amino acids situated in positions 422 and 624 of SEQ ID NO 37,
the fragment of 187 amino acids delimited by the amino acids situated in positions 447 and 633 of SEQ ID NO 37,
the fragment of 178 amino acids delimited by the amino acids situated in positions 447 and 624 of SEQ ID NO 37,
the following fragments of the WASP homologous protein (Wsp1p) of Schizosaccharomyces pombe:
the fragments of which the N-terminal amino acid corresponds to that situated in one of positions 477 to 501 of SEQ ID NO 38, and the C-terminal amino acid corresponds to that situated in one of positions 565 to 574 of SEQ ID NO 38,
the fragment of 98 amino acids delimited by the amino acids situated in positions 477 and 574 of SEQ ID NO 38,
the fragment of 89 amino acids delimited by the amino acids situated in positions 477 and 565 of SEQ ID NO 38,
the fragment of 74 amino acids delimited by the amino acids situated in positions 501 and 574 of SEQ ID NO 38,
the fragment of 65 amino acids delimited by the amino acids situated in positions 501 and 565 of SEQ ID NO 38,
or the peptide sequences derived from the abovementioned peptide fragments, in particular by substitution, addition or suppression of one or more amino acids of these fragments, said derived sequences having the property defined in claim 11 of the WASP family proteins and of said fragments of the latter.

14. Process according to one of claims 11 to 13, applied to the detection or the screening of molecules:
likely to be able to be used as medicaments in the treatment of pathologies linked to a dysfunction of the process of actin polymerisation within the scope of the formation of the actin cytoskeleton, in particular as medicaments in the treatment of metastatic cancers, or as anti-parasitic antibiotics,
or likely to have a cytotoxic effect corresponding to an inhibition or a stimulation of the formation of the actin cytoskeleton.

15. Kit for the implementation of a process according to one of claims 10 to 13, comprising
a reagent according to claim 8 or 9,
if appropriate a reagent comprising WASP family proteins in the eucaryotic cells, or peptide sequences derived from the WASP family proteins or peptide fragments defined in one of claims 11 to 13, bound or adsorbed to a support as defined in claim 8 or 9,
if appropriate a labelled compound making it possible to visualize actin polymerisation, in particular actin labelled by fluorescence,
if appropriate a suitable medium containing the elements necessary for actin polymerisation, in particular an extract of lysed cells.

16. Peptide sequences SEQ ID NO 4, SEQ ID NO 6, SEQ ID NO 20, SEQ ID NO 22, SEQ ID NO 24, SEQ ID NO 26, and SEQ ID NO 30, as well as the peptide sequences derived from the abovementioned peptide fragments, as defined in claim 6.

17. Nucleotide sequences coding for the peptide sequences according to claim 16, and corresponding to the following nucleotide sequences:
the sequence SEQ ID NO 3 coding for SEQ ID NO 4, the sequence SEQ ID NO 5 coding for SEQ ID NO 6, the sequence SEQ ID NO 19 coding for SEQ ID NO 20, the sequence SEQ ID NO 21 coding for SEQ ID NO 22, the sequence SEQ ID NO 23 coding for SEQ ID NO 24, the sequence SEQ ID NO 25 coding for SEQ ID NO 26, the sequence SEQ ID NO 29 coding for SEQ ID NO 30,
the nucleotide sequences derived by degeneration of the genetic code of the abovementioned nucleotide sequences, and coding for the abovementioned peptide sequences,
the nucleotide sequences derived from the abovementioned nucleotide sequences, and coding for the sequences derived from said peptide sequences as defined above.
